(19) United States
(12) Patent Application Publication
(10) Pub. No.: US 2010/0249084 A1
(43) Pub. Date: Sep. 30, 2010

(54) SUBSTITUTED PYRIMIDINES AS ADENOSINE RECEPTOR ANTAGONISTS

(76) Inventors: Youngsheng Chen, San Diego, CA (US); Manisha Moorjani, San Diego, CA (US); Deborah Slee, Cardiff, CA (US); John Tellew, La Jolla, CA (US); Xiaohu Zhang, San Diego, CA (US)

Correspondence Address: COOLEY LLP;ATTN: Patent Group, Suite 1100, 777 - 6th Street, NW, WASHINGTON, DC, 20001, US

(21) Appl. No.: 12/532,093

(22) PCT Filed: Mar. 21, 2008
(86) PCT No.: PCT/US08/57911

§ 371 Date: Apr. 27, 2010

(30) Foreign Application Priority Data
Mar. 21, 2007 (US) 60896198

(51) Int. Cl. A61K 31/506; C07D 401/14; C07D 413/14; A61K 31/5377; A61K 31/553; A61P 1/00; A61P 11/06; A61P 3/10; A61P 3/04; A61P 25/16; A61P 25/28

(52) U.S. Cl. 514/210.2; 544/324; 514/275; 544/122; 514/235.8; 540/544; 514/211.15

(57) ABSTRACT

Compounds of formula (I): wherein R₁, R₂ and R₃ are as defined herein, including pharmaceutically acceptable salt, ester, solvate or stereoisomer thereof. Also disclosed are compositions containing a compound of this invention in combination with a pharmaceutically acceptable carrier, as well as methods relating to the use thereof.

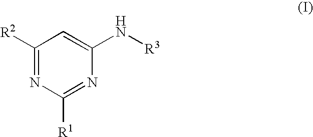

### BACKGROUND OF THE INVENTION

1. Field of the Invention

The present invention relates to adenosine receptor antagonists, in particular antagonists of the A_{2A} adenosine receptor subtype, pharmaceutical compositions comprising said compounds, and the use of said compounds in the treatment of diseases and disorders susceptible of being ameliorated by antagonism of adenosine receptors. Such diseases and disorders include movement disorders, such as Parkinson's disease, restless leg syndrome, Alzheimer's disease and dyskinesia.

2. Description of the Related Art

The effects of adenosine are mediated through at least four specific cell membrane receptors so far identified and classified as receptors A₁, A_{2A}, A_{2B} and A₃ belonging to the G protein-coupled receptor family. The A₁ and A₃ receptors down-regulate cellular cAMP levels through their coupling to G proteins, which inhibit adenylate cyclase. In contrast, A_{2A} and A_{2B} receptors couple to G proteins that activate adenylate cyclase and increase intracellular levels of cAMP. Through these receptors, adenosine regulates a wide range of physiological functions.

Thus, in the cardiovascular system the activation of the A₁ receptor protects cardiac tissue from the effects of ischemia and hypoxia. A similar protective effect is also produced by antagonism of the A_{2A} receptor, which enhances A₁-receptor-induced antiadrenergic responses and may also be useful in the treatment of acute myocardial ischemia and supraventricular arrhythmias (Norton G R et al. Am J Physiol. 1999; 276(2 Pt 2):H341-9; Auchampach J A, Bolli R. Am J Physiol. 1999; 276(3 Pt 2):H1113-6). In addition, the A_{2B} adenosine receptor subtype (Feoktistov, I. et al., Pharmacol. Rev. 1997, 49, 381-402) appears to be involved in the control of vascular tone and the regulation of vascular smooth muscle growth.

In the kidney, adenosine exerts a biphasic action, inducing vasodilation at high concentrations and vasoconstriction at low concentrations. Thus, adenosine plays a role in the pathogenesis of some forms of acute renal failure that may be ameliorated by A₁ receptor antagonists (Costello-Boerrigter L C, et al. Med Clin North Am. 2003 March; 87(2): 475-91; Gottlieb S S., Drugs. 2001; 61(10): 1387-93).

Adenosine is also involved in the physiopathology of the immune system. It can induce degranulation of activated human mast cells through the A_{2B} and/or A₃ receptor. Thus A_{2B} and/or A₃ antagonists prevent mast cell degranulation and are, therefore, useful in the treatment, prevention or suppression of disease states induced by activation of the A_{2B} and/or A₃ receptor and mast cell degranulation. These disease states include but are not limited to asthma, myocardial reperfusion injury, allergic reactions including but not limited to rhinitis, urticaria, scleroderm arthritis, other autoimmune diseases and inflammatory bowel diseases.

Furthermore, in the respiratory system adenosine induces bronchoconstriction, modulates airway inflammation and promotes neutrophil chemotaxis. Therefore, an adenosine antagonist would be particularly useful in the treatment of asthma.

In the gastrointestinal and metabolic system, the A_{2B} adenosine receptor subtype (Feoktistov, I. et al., Pharmacol. Rev. 1997, 49, 381-402) seems to be involved in the regulation of hepatic glucose production, the modulation of intestinal tone, as well as intestinal secretion. Thus, A_{2B} antagonists may also be useful in the treatment of diabetes mellitus and obesity.

In the central nervous system adenosine is a potent endogenous neuromodulator, which controls the presynaptic release of many neurotransmitters and is thus involved in motor function, sleep, anxiety, pain and psychomotor activity. All adenosine receptor subtypes are present in the brain, with A₁ and A_{2A} subtypes being differentially distributed. The former are found predominantly in the hippocampus and cortex, whilst the latter are found mainly in the striatum. Adenosine A_{2A} receptors modulate the release of GABA in the striatum, which possibly regulates the activity of medium spiny neurons.

Thus, A_{2A} receptor antagonists may be a useful treatment for neurodegenerative movement disorders such as Parkinson and Huntington's disease (Tuite P, et al., J. Expert Opin Investig Drugs. 2003; 12: 1335-52; Popoli P. et al. J Neurosci. 2002; 22:1967-75), dystonias such as restless leg syndrome (Happe S, et al., Neuropsychobiology. 2003; 48: 82-6), Alzheimer's disease (Dall'Igna, et al., Experimental Neurology, 2007; 241-245) and dyskinesias such as those caused by prolonged use of neuroleptic and dopaminergic drugs (Jenner P. J Neurol. 2000; 247 Suppl2: 1143-50).

In the treatment of Parkinson's disease an A_{2A} antagonist may be useful not only as monotherapy, but also when administered in combination with L-DOPA and/or one or more of the following drugs: dopamine agonists, inhibitors of dopamine decarboxylase, catechol-O-methyltransferase inhibitors and inhibitors of monoamine oxidase.

In addition, A_{2A} antagonists may have therapeutic potential as neuroprotectants (Stone T W. et al., Drug. Dev. Res. 2001; 52: 323-330), and in the treatment of sleep disorders (Dunwiddie T V et al., Ann. Rev. Neurosci. 2001; 24: 31-55).

It has now been found that certain 4-aminopyrimidine derivatives are novel potent antagonists of the A_{2A} adenosine receptor and can therefore be used in the treatment or prevention of diseases susceptible to amelioration by antagonism of the adenosine receptor.

Further objectives of the present invention are to provide a method for preparing said compounds; pharmaceutical compositions comprising an effective amount of said compounds; the use of the compounds in the manufacture of a medicament for the treatment of pathological conditions or diseases susceptible of being improved by antagonism of an adenosine receptor, in particular by antagonism of the A_{2A} adenosine receptor; methods of treatment of pathological conditions or diseases susceptible to amelioration by antagonism of an adenosine receptor, in particular by antagonism of the A_{2A} adenosine receptor comprising the administration of the compounds of the invention to a subject in need of treatment and combinations of said compounds with one or more of the following drugs: L-DOPA, dopamine agonists, inhibitors of dopamine decarboxylase, catechol-O-methyltransferase inhibitors and inhibitors of monoamine oxidase.

### BRIEF SUMMARY OF THE INVENTION

In brief, this invention is generally directed to adenosine receptor antagonists, as well as to methods for their preparation and use, and to pharmaceutical compositions containing the same. More specifically, the adenosine receptor antagonists of this invention are compounds having the following general structure (I):

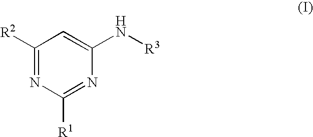

and pharmaceutically acceptable salts, esters, solvates and stereoisomers thereof, wherein R¹, R² and R³ are as defined below.

The compounds of this invention may generally be used to treat a variety of disorders or conditions, particularly those which benefit from inhibition of adenosine (particularly A_{2A}) receptors. Accordingly, in another embodiment, methods are disclosed for treating one or more of a variety of diseases or conditions, including (but not limited to) ischemia, supraventricular arrhythmias, acute renal failure, myocardial reperfusion injury, autoimmune disease, addiction, substance abuse, excessive daytime sleepiness, inflammatory bowel diseases, asthma, diabetes mellitus, obesity, Parkinson disease, Huntington's disease, Alzheimer's disease, dystonia or dyskinesia.

The methods of this invention generally involve administering an effective amount of one or more compounds of this invention, typically in the form of a pharmaceutical composition, to an animal (also referred to here as a “patient”, including a human) in need thereof. Accordingly, in still another embodiment, compositions are disclosed containing one or more compounds of this invention and a pharmaceutically acceptable carrier and/or diluent.

These and other aspects of the invention will be apparent upon reference to the following detailed description. To that end, various references are set forth herein which describe in more detail certain procedures, compounds and/or compositions, and are hereby incorporated by reference in their entirety.

### DETAILED DESCRIPTION OF THE INVENTION

As mentioned above, the present invention is directed generally to compounds useful as adenosine receptor antagonists. The compounds of this invention have the following structure (I):

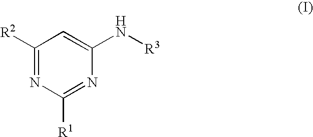

and pharmaceutically acceptable salts, esters, solvates and stereoisomers thereof, wherein:

R¹ is a heterocycle optionally substituted by one or more members selected from the group of lower alkyl, lower alkoxy, halogen and cyano;

R² is phenyl or pyridyl, wherein the phenyl or pyridyl ring is substituted by 1 to 4 R⁴ groups; or

R² is isoquinoline, dihydroisoquinoline or tetrahydroisoquinoline ring, wherein the isoquinoline, dihydroisoquinoline or tetrahydroisoquinoline ring is optionally substituted by R⁵;

R³ is H, R⁷, COR⁷, CONR⁷R⁸, or COOR⁷;

R⁴ is at each occurrence selected from the group of halogen, —(X), —(O)ₙ—(Y)ₚ—R⁵, or —(X)ₘ—(O)ₙ—(Y)ₚ—NR⁵R⁶;

each of R⁵ and R⁶ is independently hydrogen, lower alkyl, lower C₂₋₆ alkoxy, lower C₂₋₆ alkoxyalkyl, lower C₂₋₆ hydroxyalkyl, cyano, C(O)—C₁-C₆alkyl or C(O)O—C₁-C₆alkyl; or

R⁵ and R⁶ together with the nitrogen to which they are attached form a heterocyclic ring optionally substituted by one or more members selected from the group of halogen, hydroxyl, lower alkyl, lower alkoxy, lower alkoxyalkyl, lower hydroxyalkyl, cyano, and —C(O)—C₁-C₆alkyl;

R⁷ is lower alkyl optionally substituted by one or more members selected from the group of lower alkyl, lower alkoxy, hydroxyl, halogen, amino, alkylamino, and dialkylamino;

R⁸ is selected from the group of lower alkyl, lower alkoxy, alkoxyalkyl, —C(O)—C₁-C₆alkyl or lower alkenyl, wherein the lower alkyl, lower alkoxy, alkoxyalkyl, —C(O)—C₁-C₆alkyl, and lower alkenyl groups are optionally substituted by one or more lower alkyl, halogen, lower alkoxy, hydroxyl, or cyano; or

R⁷ and R⁸ together with the nitrogen to which they are attached form a heterocyclic ring optionally substituted by one or more members selected from the group of halogen, hydroxyl, lower alkyl, lower alkoxy, lower alkoxyalkyl, lower hydroxyalkyl, and cyano;

each of X and Y is independently lower alkyl, cycloalkyl or saturated heterocyclyl;

m is at each occurrence 0 or 1;

n is at each occurrence 0 or 1; and

p is at each occurrence 0 or 1.

Other aspects of the present invention are: a) pharmaceutical compositions containing a pharmaceutically effective amount of said compounds, b) the use of said compounds in the manufacture of a medicament for the treatment of diseases susceptible of being improved by antagonism of an adenosine receptor, in particular by antagonism of the A_{2A} adenosine receptor; c) methods of treatment of diseases susceptible to amelioration by antagonism of an adenosine receptor, in particular by antagonism of the A_{2A} adenosine receptor, which methods comprise the administration of the compounds of the invention to a subject in need of treatment, and administration of combinations of said compounds with one or more of the following drugs: L-DOPA, dopamine agonists, inhibitors of dopamine decarboxylase, catechol-β-methyltransferase inhibitors and inhibitors of monoamine oxidase.

As used herein the term lower alkyl embraces optionally substituted, linear or branched alkyl radicals having 1 to 8 carbon atoms. Typically lower alkyl groups have 1 to 6 or 1 to 4 carbon atoms. Typical examples of substituents in said alkyl groups are halogen, hydroxy and amino.

Examples of lower alkyl groups include methyl, ethyl, n-propyl, propyl, n-butyl, sec-butyl and tert-butyl, n-pentyl, 1-methylbutyl, 2-methylbutyl, isopentyl, 1-ethylpropyl, 1,1-dimethylpropyl, 1,2-dimethylpropyl, n-hexyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl, 2-methylpentyl, 3-methylpentyl and iso-hexyl radicals.

As used herein, the term lower alkoxy embraces optionally substituted, linear or branched oxy-containing radicals each having alkyl portions of 1 to 8, typically 1 to 6 and more typically 1 to 4 carbon atoms. Typical examples of substituents in said alkoxy groups are halogen, hydroxy and amino.

Examples of lower alkoxy groups include methoxy, ethoxy, n-propoxy, i-propoxy, n-butoxy, sec-butoxy, t-butoxy, trifluoromethoxy, difluoromethoxy, hydroxymethoxy, 2-hydroxyethoxy or 2-hydroxypropoxy.

As used herein, the term lower alkylthio embraces radicals containing an optionally substituted, linear or branched alkyl radicals of 1 to 8, typically 1 to 6 and more typically 1 to 4 carbon atoms. Typical examples of substituents in said alkoxy groups are halogen, hydroxy and amino.

Examples of optionally substituted lower alkylthio radicals include methylthio, ethylthio, n-propylthio, i-propylthio, n-butylthio, sec-butylthio, t-butylthio, trifluoromethylthio, difluoromethylthio, hydroxymethylthio, 2-hydroxyethylthio or 2-hydroxypropylthio.

As used herein the term “acyl” refers to groups represented by the formula alkyl-C(═O)—, where the alkyl group may be substituted or unsubstituted.

As used herein, the term cyclic group embraces, unless otherwise specified, carbocyclic and heterocyclic radicals. The cyclic radicals can contain one or more rings. Carbocyclic radicals may be aromatic or alicyclic, for example cycloalkyl radicals. Heterocyclic radicals also include heteroaryl radicals.

As used herein, the term aromatic group embraces typically a 5- to 14-membered aromatic ring system, such as a 5- or 6-membered ring which may contain one or more heteroatoms selected from O, S and N. When no heteroatoms are present the radical is named aryl radical and when at least one heteroatom is present it is named heteroaryl radical. The aromatic radical can be monocyclic or polycyclic, such as phenyl or naphthyl. When an aromatic radical or moiety carries 2 or more substituents, the substituents may be the same or different.

As used herein, the term aryl radical embraces typically a C₅-C₁₄ monocyclic or polycyclic aryl radical such as phenyl, naphthyl, anthranyl or phenanthryl. When an aryl radical carries 2 or more substituents, the substituents may be the same or different.

As used herein, the term heteroaryl radical embraces typically a 5- to 14-membered ring system comprising at least one heteroaromatic ring and containing at least one heteroatom selected from O, S and N. A heteroaryl radical may be a single ring or two or more fused rings wherein at least one ring contains a heteroatom.

Examples of heteroaryls include pyridyl, pyrazinyl, pyrimidinyl, pyridazinyl, furyl, oxadiazolyl, oxazolyl, isoxazolyl, imidazolyl, thiazolyl, thiadiazolyl, thienyl, pyrrolyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl and pyrazolyl. When a heteroaryl radical carries 2 or more substituents, the substituents may be the same or different.

As used herein, the term heterocycle radical embraces typically a 5- to 14-membered ring system comprising at least one heterocyclic ring and containing at least one heteroatom selected from O, S and N. A heterocycle radical may be a single ring or two or more fused rings wherein at least one ring contains a heteroatom. A heterocycle radical may be aromatic, in which case it is a heteroaryl radical, or it may be non-aromatic.

Examples of aromatic heterocycles (i.e., heteroaryls) are provided above. Examples of non-aromatic heterocycles include piperidinyl, piperazinyl, morpholinyl, pyrrolidinyl, thiomorpholinyl, oxazolidinyl, imidazolidinyl, thiazolidinyl, azepanyl, [1,4]diazepanyl, [1,4]oxazepanyl and thiazepanyl.

As used herein, the term cycloalkyl embraces saturated optionally substituted carbocyclic radicals and, unless otherwise specified, a cycloalkyl radical typically has from 3 to 7 carbon atoms. The preferred substituents in said cycloalkyl groups are selected from halogen atoms, hydroxy groups, alkyl groups and amino groups.

Examples include cyclopropyl, cyclobutyl, cyclopentyl, cyclohexyl and cycloheptyl. It is preferably cyclopropyl, cyclopentyl or cyclohexyl. When a cycloalkyl radical carries 2 or more substituents, the substituents may be the same or different.

As used herein, some of the atoms, radicals, moieties, chains or cycles present in the general structures of the invention are “optionally substituted”. This means that these atoms, radicals, moieties, chains or cycles can be either unsubstituted or substituted in any position by one or more, for example 1, 2, 3 or 4, substituents, whereby the hydrogen atoms bound to the unsubstituted atoms, radicals, moieties, chains or cycles are replaced by chemically acceptable atoms, radicals, moieties, chains or cycles. When two or more substituents are present, each substituent may be the same or different.

The substituents of an “optionally substituted” structure may include, without limitation, one or more, typically one to four, and more typically one to two of the following substituents: alkyl, alkenyl, alkynyl, aryl, heteroaryl, alkoxy, aryloxy, alkylthio, arylthio, cycloalkyl, arylalkyl, amino, alkylamino, dialkylamino, amido (e.g. CONH2, CONHalkyl and CONHdialkyl and reverse NCOH or NCOalkyl), F, Cl, Br, I, CN, NO₂, NH₂, NHCH₃, NHCH₂CH₃, N(CH₃)₂, N(CH₂CH₃)₂, SH, SCH₃, OH, OCH₃, OCF₃, CH₃, and CF₃.

As used herein, the term halogen atom embraces chlorine, fluorine, bromine or iodine atoms typically a fluorine, chlorine or bromine atom, most preferably chlorine or fluorine. The term halo when used as a prefix has the same meaning.

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

Other preferred salts according to the invention are quaternary ammonium compounds wherein an equivalent of an anion (X⁻) is associated with the positive charge of the N atom. X⁻ may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X⁻ is preferably an anion selected from chloride, bromide, iodide, sulphate, nitrate, acetate, maleate, oxalate, succinate or trifluoroacetate. More preferably X⁻ is chloride, bromide, trifluoroacetate or methanesulphonate.

As used herein, an N-oxide is formed from the tertiary basic amines or imines present in the molecule, using a convenient oxidising agent.

In one embodiment of the present invention in the compounds of formula (I), R¹ represents a heterocycle optionally substituted by one or more members selected from the group of lower alkyl, lower alkoxy, halogen and cyano.

According to one embodiment of the present invention, R¹ represents a heteroaryl group selected from the group of pyridinyl, furanyl, thiophenyl, thiazolyl, pyrazolyl, triazolyl, imidiazolyl, oxazolyl, isoxazolyl and oxadiazolyl groups which are optionally substituted by one or more substituents selected from the group of lower alkyl, lower alkoxy, halogen and cyano.

In one embodiment of the present invention R¹ represents a heteroaryl group selected from the following:

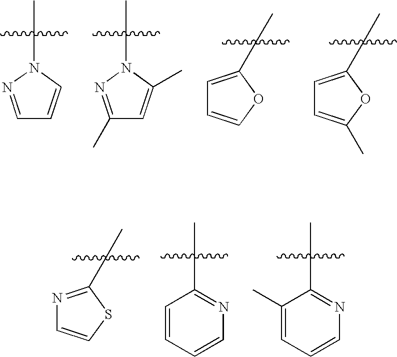

In one embodiment of the present invention in the compounds of formula (I), R² represents a phenyl or pyridyl group substituted by 1 to 4 R⁴ groups.

In one embodiment of the present invention where R² is pyridyl, R⁴ is a member selected from the group of pyrrolidinyl, morpholinyl, piperidinyl, oxazepanyl, piperazinyl, azetidinyl, alkylamino, dialkylamino and dialkylamino, wherein the pyrrolidinyl, morpholinyl, piperidinyl, oxazepanyl, piperazinyl and azetidinyl are optionally substituted by one or more lower alkyl, lower alkoxy or hydroxyl. In one such embodiment of the present invention R² represents a pyridyl substituted by a group selected from the following:

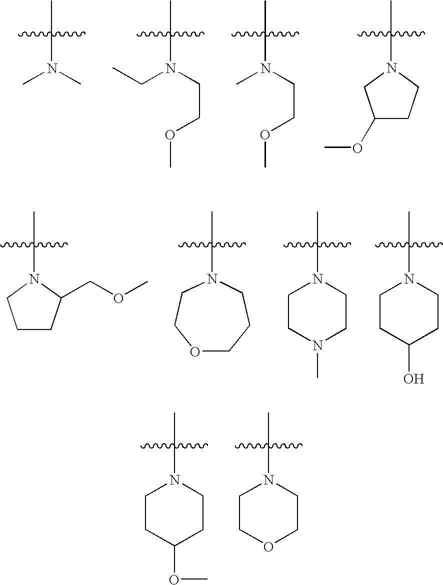

In one embodiment of the present invention where R² is phenyl, R⁴ is a heterocyclic group linked to the phenyl ring through an oxygen atom. In one such embodiment of the present invention R² represents phenyl substituted by a group selected from the following:

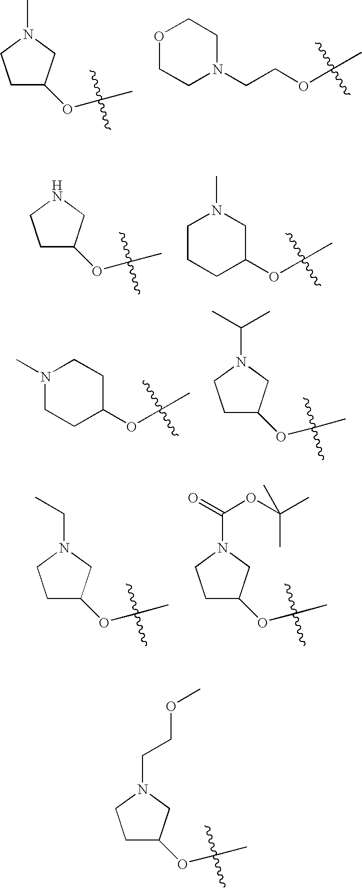

In such embodiment of the present invention, the phenyl group may be further substituted by one or more additional R⁴ groups such as, for example, halogen.

In one embodiment of the present invention where R² is phenyl, R⁴ is a heterocyclic group linked to the phenyl ring or through a carbon atom. In one such embodiment of the present invention R² represents phenyl substituted by a group selected from the following:

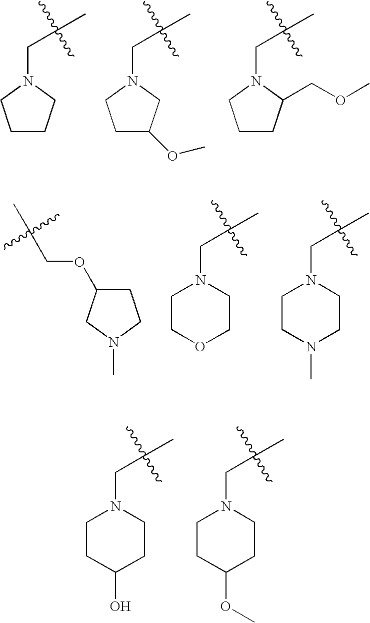

In another embodiment the heterocyclic group is an optionally substituted tetrahydroisoquinoline. In one such embodiment of the present invention R² represents a group selected from the following:

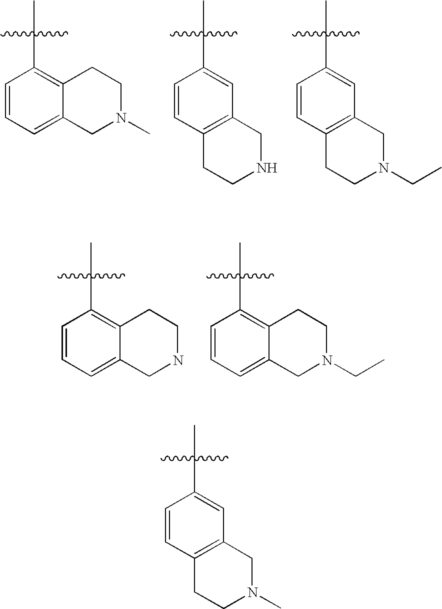

According to still another embodiment of the present invention in the compounds of formula (I), R³ represents a hydrogen, acyl, alkyl, alkoxyl, alkyloxycarbonyl, or heteroaryclyl group. In one embodiment, R³ is hydrogen or acyl.

In one embodiment of the present invention R³ represents a group selected from the following:

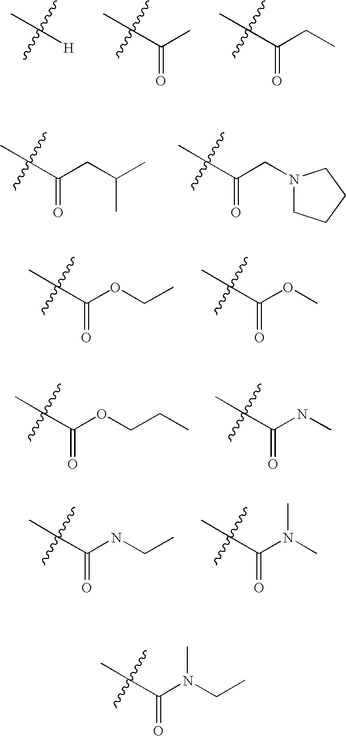

The compounds of the present invention may be prepared by one of the processes described below.

Compounds of formula (I), and in particular those of formulas (VIII) or (IX) where R¹ is a monocyclic or polycyclic heteroaryl group linked to the pyrimidine ring through a carbon atom and R² is a substituted phenyl or pyridyl group, can be obtained as shown is Scheme 1.

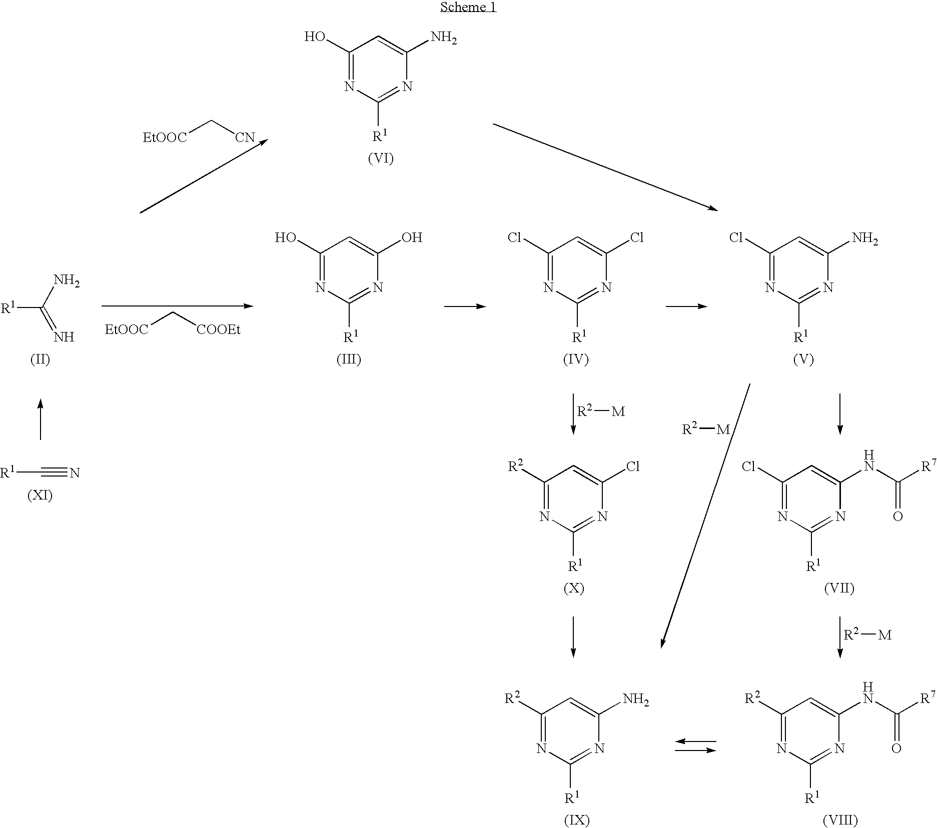

The carboxyamidines of formula (II), wherein R¹ is a monocyclic or polycyclic heteroaryl group linked to the carboxyamidine group through a carbon atom can be obtained by reacting a nitrile of formula (XI) with trimethylaluminum and ammonium chloride, in a solvent such as benzene, toluene or xylene, at a temperature from 80° C. to 120° C. It also can be obtained by reaction of a nitrile of formula (XI) with sodium methoxide in methanol at room temperature, followed by reaction with ammonium chloride at the same temperature.

The carboxyamidines of formula (II) can be reacted with diethyl malonate in a solvent such as methanol, ethanol, isopropyl alcohol, butyl alcohol or tetrahydrofuran, in the presence of a base, such as sodium methoxide, sodium ethoxide or potassium tertbutoxide and at a temperature from room temperature to the boiling point of the solvent to yield the pyrimidine-4,6-diols of formula (III).

The resulting pyrimidine-4,6-diols of formula (III) can be reacted with a chlorinating agent such a phosphorus oxychloride, phosphorus pentachloride or a mixture of them, in a solvent such as phosphorus oxychloride, benzene or toluene, at a temperature from room temperature to the boiling point of the solvent to yield the 4,6-dichloropyrimidine compounds of formula (IV). Optionally, the presence of a base such as dimethylaniline, triethylamine or diisopropyl-ethylamine may be needed in this reaction step.

The reaction of the 4,6-dichloropyrimidine compounds of formula (IV) with ammonia in a solvent such as water, methanol, ethanol, isopropyl alcohol or tetrahydrofuran, at a temperature from 80° C. to 140° C. produces the 6-chloropyrimidin-4-amines of formula (V).

Compounds of formula (V) may also be obtained by reacting a compound of formula (VI) with a chlorinating agent such as phosphorus oxychloride, phosphorus pentachloride or a mixture of them, in a solvent such as phosphorus oxychloride, benzene or toluene, at a temperature from room temperature to the boiling point of the solvent. Optionally, the presence of a base such as dimethylaniline, triethylamine or diisopropyl-ethylamine may be needed in this reaction step.

The 6-aminopyrimidin-4-ol compounds of formula (VI) are in turn obtained by reaction of the carboxyamidines of formula (II) with ethyl cyanoacetate. The reaction is carried out in a solvent such as methanol, ethanol, isopropyl alcohol, butyl alcohol or tetrahydrofuran, in the presence of a base, such as sodium methoxide, sodium ethoxide or potassium tertbutoxide and at a temperature from room temperature to the boiling point of the solvent.

The 6-chloropyrimidin-4-amines of formula (V) can be acylated by an acid chloride and a base, such as pyridine, triethylamine or diisopropylethylamine, in a solvent such as tetrahydrofuran, methylene chloride, chloroform or pyridine, at a temperature from room temperature to the boiling point of the solvent to yield the compounds of formula (VII). Compounds of formula (VII) can also be prepared by reaction of amine (V) with an anhydride, at a temperature from 80° C. to 160° C.

Compounds of formula (VII) are reacted with a compound of formula R²-M wherein R² is a substituted pyridyl or phenyl group to yield the compounds of formula (VIII), a specific case of formula (I). M is a suitable metal such as boron, tin, silicon, or zinc, in each case substituted with additional ligands as appropriate. Suitable ligated metallic groups include boronic acid or ester. The reaction is carried out in a solvent such as dioxane, tetrahydrofuran, dimethylformamide, dimethylacetamide or toluene, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C.

The compounds of formula (VIII) can be converted to compounds of formula (IX) according to the invention by reaction with sodium, potassium, or lithium hydroxide in a solvent such as methanol, ethanol, isopropyl alcohol, water or tetrahydrofuran, at a temperature from 50° C. to 100° C.

Alternatively, the compounds of formula (IX) according to the invention can also be obtained from the compounds of formula (VIII) by reaction with a mineral acid, such as hydrochloric acid or sulphuric acid, in a solvent such as water, methanol, ethanol or isopropyl alcohol, at a temperature from room temperature to the boiling point of the solvent.

The reversal of the above reaction is also possible; that is, compounds of formula (IX) can be acylated by an acid chloride and a base, such as pyridine, triethylamine or diisopropylethylamine, in a solvent such as tetrahydrofuran, methylene chloride, chloroform or pyridine, at a temperature from room temperature to the boiling point of the solvent to yield the compounds of formula (VIII). Compounds of formula (VIII) can also be prepared by reaction of a compound of formula (IX) with an anhydride, at a temperature from 80° C. to 160° C.

The compounds of formula (IX) can also be obtained directly from compounds of formula (V) by reaction with a compound of formula R²-M, wherein R² is a substituted benzene or pyridyl group. M is a suitable metal such as boron, tin, silicon, or zinc, in each case substituted with additional ligands as appropriate. Suitable ligated metallic groups include boronic acid or ester. The reaction is carried out in a solvent such as tetrahydrofuran, toluene, dioxane, dimethylformamide, or dimethylacetamide, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C.

The compounds of formula (IX) can also be obtained by reaction of chloropyrimidine compounds of formula (X) with ammonia in a solvent such as water, methanol, ethanol, isopropyl alcohol or tetrahydrofuran, at a temperature from 80° C. to 140° C.

The compounds of formula (X) are in turn obtained by selective mono-coupling of the dichloropyrimidine compound of formula (IV) with a compound of formula R²-M, wherein R² is a substituted phenyl or pyridyl group. M is a suitable metal such as boron, tin, silicon, or zinc, in each case substituted with additional ligands as appropriate. Suitable ligated metallic groups include boronic acid or ester. The reaction is carried out in a solvent such as tetrahydrofuran, dioxane, dimethylformamide, dimethylacetamide or dimethylsulfoxide, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C.

Compounds of formula (I), and in particular those of formulas (XIII) or (XV) where R^{1′} and R¹″ are H or small alkyl and X is N or a carbon optionally substituted by a small alkyl or halogen, can be obtained as shown is Scheme 2.

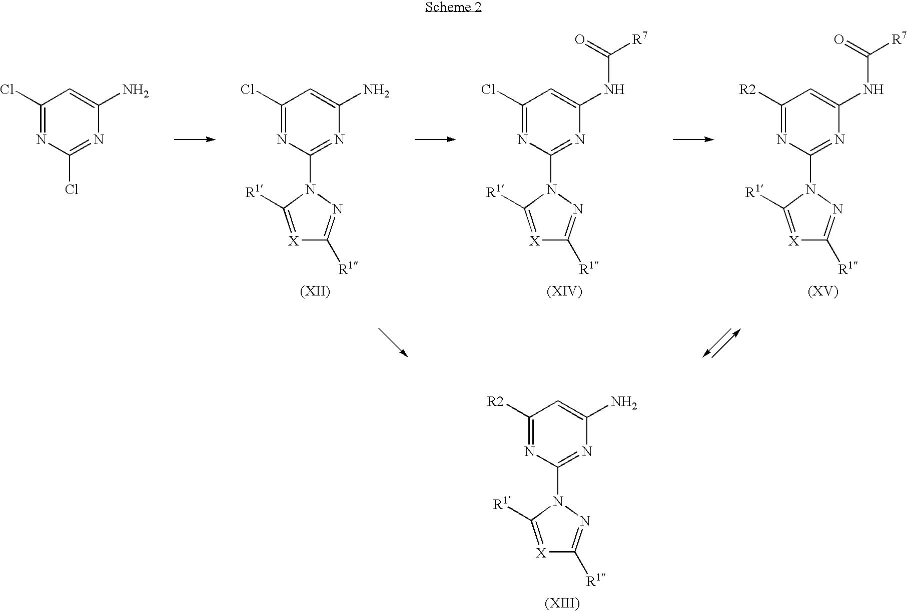

The compounds of formula (XII) can be obtained from 2,4-dichloro 6-aminopyrimidine by reaction with anhydrous hydrazine in the presence of a solvent such as ethanol or NMP at a temperature of room temperature to 80° C., then by reacting the intermediate with the appropriate dicarbonyl compound at a temperature from room temperature to 100° C.

The compounds of formula (XIV) can be obtained from the 6-chloropyrimidine-4-amines compounds of formula (XII) by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (XIV) can also be prepared by reaction of amine (XII) with an anhydride, at a temperature from 80° C. to 160° C.

The resulting 6-chloropyrimidin-4-amines of formula (XIV) are reacted with a compound of formula R²-M, wherein R² is a substituted phenyl or pyridyl group, to provide compounds of formula (XV), which is a particular case of the compounds of formula (I) according to the invention. M is a suitable metal such as boron, tin, silicon, or zinc, in each case substituted with additional ligands as appropriate. Suitable ligated metallic groups include boronic acid or ester. The reaction is carried out in a solvent such as tetrahydrofuran, dioxane, dimethylformamide, dimethylacetamide or toluene, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C.

The compounds of formula (XV) can also be obtained from the 4-aminopyrimidine compounds of formula (XIII) by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (XV) can also be prepared by reaction of a compound of formula (XIII) with an anhydride, at a temperature from 80° C. to 160° C.

The amine compounds of formula (XIII), which are a particular case of compounds of formula (I), can be obtained by reaction of compounds of formula (XII) with a compound of formula R²-M, wherein R² is a substituted phenyl or pyridyl group. M is a suitable metal such as boron, tin, silicon, or zinc, in each case substituted with additional ligands as appropriate. Suitable ligated metallic groups include boronic acid or ester. The reaction is carried out in a solvent such as tetrahydrofuran, dioxane, dimethylformamide, dimethylacetamide or toluene, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C.

Alternatively, the amine compounds of formula (XIII) may be obtained by hydrolysis of compounds of formula (XV) by reaction with sodium, potassium, or lithium hydroxide in a solvent such as methanol, ethanol, isopropyl alcohol, water or tetrahydrofuran, at a temperature from 50° C. to 100° C. The hydrolysis can also be performed with a mineral acid, such as hydrochloric acid or sulphuric acid, in a solvent such as water, methanol, ethanol or isopropyl alcohol, at a temperature from room temperature to the boiling point of the solvent.

Compounds of formula (I) and in particular those of formulas (XVIII) where R^{1′} and R¹″ are H or small alkyl and X is a nitrogen or a carbon optionally substituted by small alkyl or halogen, can be obtained as shown is Scheme 3.

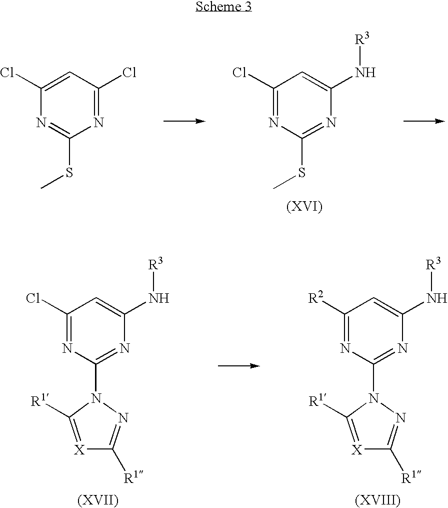

When R³ is COR^{E}, compounds of formula (XVI) can be prepared by reaction of 4,6-dichloro-2-(methylthio)pyrimidine with ammonia in a solvent such as water, methanol, ethanol, isopropyl alcohol or tetrahydrofuran, at a temperature from 80° C. to 140° C. followed by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (XVI) can also be prepared by reaction of the 4,6-dichloro 2-methylthiolpyrimidine with ammonia in a solvent such as water, methanol, ethanol, isopropyl alcohol or tetrahydrofuran, at a temperature from 80° C. to 140° C. followed by reaction with an anhydride, at a temperature from 80° C. to 160° C.

The compounds of formula (XVII) can be obtained by oxidation of a compound of formula (XVI) to the sulfone in presence of an oxidazing reagent such as OXONE®, hydrogen peroxide, potassium permanganate, a peracid, or sodium perborate. The sulfone intermediate is then reacted with anhydrous hydrazine in the presence of a solvent such as NMP at a temperature of 60° C. and reacted with the appropriate dicarbonyl compound at a temperature from room temperature to 60° C.

The resulting 6-chloropyrimidines of formula (XVII) are reacted with a compound of formula R²-M, wherein R² is a substituted phenyl or pyridyl group, to yield compounds of formula (XVIII) which is a particular case of the compounds of formula (I) according to the invention. M is a suitable metal such as boron, tin, silicon, or zinc, in each case substituted with additional ligands as appropriate. Suitable ligated metallic groups include boronic acid or ester. The reaction is carried out in a solvent such as tetrahydrofuran, dioxane, dimethylformamide, dimethylacetamide or toluene, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C.

Compounds of formula (I) and in particular those of formulas (XX) and (XXI) where R^{1′} and R^{1″} are H, small alkyl or halogen, can be obtained as shown in Scheme 4.

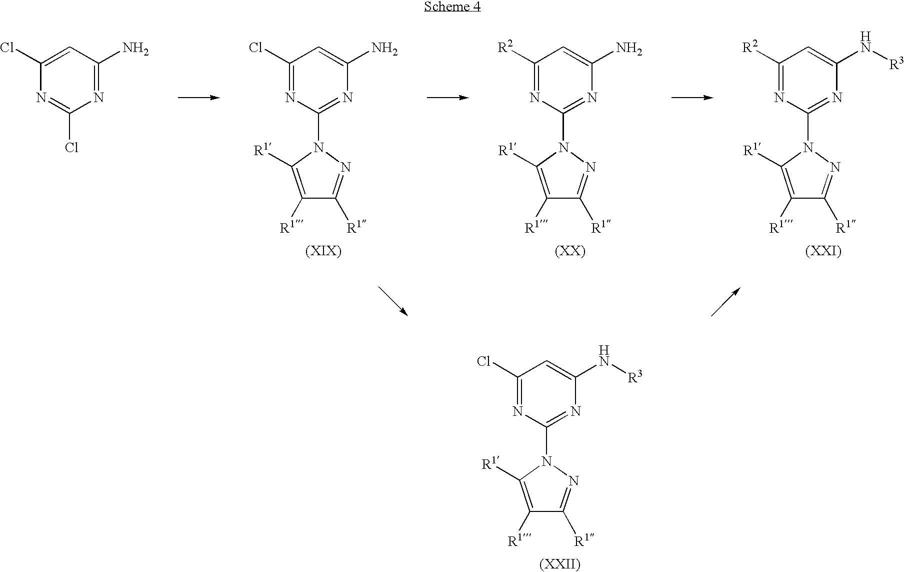

The compounds of formula (XIX) can be obtained by reacting 4-amino-2,6-dichloropyrimidine with an optionally substituted pyrazole. The reaction is carried out in a solvent such as dioxane, dimethylformamide, dimethylacetamide or dimethylsulfoxide, in the presence of a base, such as sodium hydride, potassium carbonate or cesium carbonate, at a temperature from 60° C. to 140° C.

The resulting 6-chloropyrimidin-4-amines of formula (XIX) are reacted with a compound of formula R²-M, wherein R² is a substituted phenyl or pyridyl group, to yield the compounds of formula (XX) which are a particular case of the compounds of formula (I) according to the invention. M is a suitable metal such as boron, tin, silicon, or zinc, in each case substituted with additional ligands as appropriate. Suitable ligated metallic groups include boronic acid or ester. The reaction is carried out in a solvent such as tetrahydrofuran, dioxane, dimethylformamide, dimethylacetamide or toluene, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C.

The compounds of formula (XXI) can be obtained from compounds of formula (XX) by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (XXI), which is a particular case of the compounds of formula (I) according to the invention, can also be prepared by reaction of amines of formula (XX) with an anhydride, at a temperature from 80° C. to 160° C.

The compounds of formula (XXII) can be obtained from the 6-chloropyrimidine-4-amine compounds of formula (XIX) by acylation with an acid chloride and a base, such as pyridine, triethylamine or diisopropylethylamine, in a solvent such as tetrahydrofuran, methylene chloride, chloroform or pyridine, at a temperature from room temperature to the boiling point of the solvent. Compounds of formula (XXII) can also be prepared by reaction of amine (XIX) with an anhydride, at a temperature from 80° C. to 160° C.

The resulting 6-chloro2-pyrazolopyrimidines of formula (XXII) are reacted with a compound of formula R²-M, wherein R² is a substituted phenyl or pyridyl group, to yield the compounds of formula (XXI) which is a particular case of the compounds of formula (I) according to the invention. M is a suitable metal such as boron, tin, silicon, or zinc, in each case substituted with additional ligands as appropriate. Suitable ligated metallic groups include boronic acid or ester. The reaction is carried out in a solvent such as tetrahydrofuran, dioxane, dimethylformamide, dimethylacetamide or toluene, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C.

Carbamates of formula (XXIII), which are a particular case of compounds of formula (I), can be prepared as shown in Scheme 6.

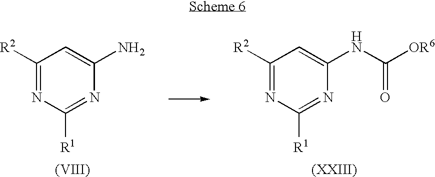

The carbamates of formula (XXIII) are obtained by reaction of a compound of formula (VIII) with a compound of formula Z—COOR⁷, wherein Z represents a leaving group such as halogen atom, preferably chlorine or a group selected from ethoxy, methoxy, p-nitrophenoxy and imidazolyl. The reaction is carried out in a solvent, such as tetrahydrofuran, chloroform, methylene chloride or dimethylformamide, in the presence of a base, preferably triethylamine, diisopropylethylamine, potassium carbonate or sodium hydroxide, at a temperature from −70° C. to 100° C.

The carbamates of formula (XXIII) can also be obtained by reaction of a compound of formula (VIII) with triphosgene or phosgene, in a solvent such as tetrahydrofuran, chloroform, or methylene chloride, in the presence of a base such as pyridine, at a temperature from −5° C. to 50° C., followed by reaction with an alcohol of formula HO—R⁶

Compounds of formula (XXIV) and (XXV), which are a particular case of compounds of formula (I) in which the phenyl group of R² is substituted by a hydroxyl or alkoxy group, can be prepared as shown in Scheme 7.

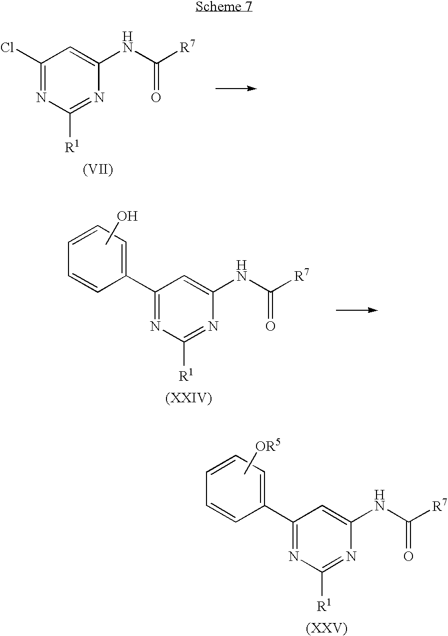

Compounds of formula (VII) are reacted with a compound of formula R²-M, wherein R² is a phenyl group substituted at a minimum with a hydroxyl group, to yield the compounds of formula (XXIV). M is a suitable metal such as boron substituted with additional ligands as appropriate. Suitable ligated metallic groups include boronic acid or ester. The reaction is carried out in a solvent such as dioxane, tetrahydrofuran, dimethylformamide, dimethylacetamide or toluene, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C.

Compounds of formula (XXIV) are reacted with an alcohol of formula HO—R⁵ to yield to compounds of formula (XXV). The reaction is carried out in a solvent such as tetrahydrofuran or toluene, in the presence of a phosphine such as triphenylphosphine and an azodicarboxylate such as diethyl azodicarboxylate or diisopropyl azodicarboxylate at ambient temperature.

Compounds of formulas (XXVII) and (XXVIII), in which one of G¹-G⁵ may be a nitrogen atom and the rest are optionally substituted carbon atoms, are special cases of compounds of formula (I) and may be prepared as shown in Scheme 8.

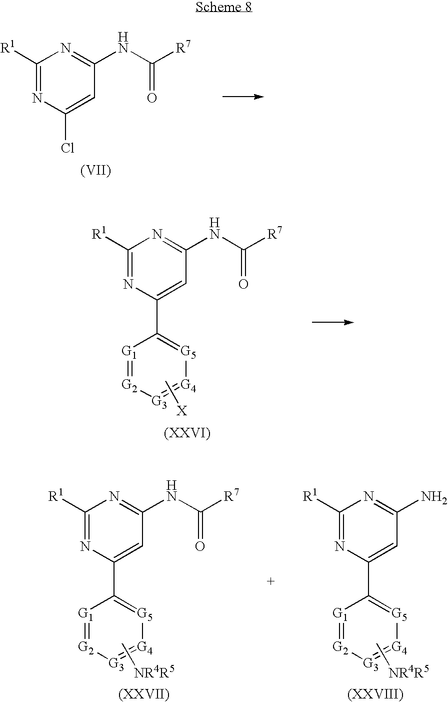

Compounds of formula (VII) are reacted with a compound of formula R²-M, wherein R² is a pyridyl or phenyl group substituted at a minimum with a halogen, to yield the compounds of formula (XXVI). M is a suitable metal such as boron substituted with additional ligands as appropriate. Suitable ligated metallic groups include boronic acid or ester. The reaction is carried out in a solvent such as dioxane, tetrahydrofuran, dimethylformamide, dimethylacetamide or toluene, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C.

In the case where one of the G groups is nitrogen, the compounds of formula (XXVI) may be converted to a mixture of the compounds of formulas (XXVII) and (XXVIII) by reaction with an amine of structure HNR⁴R⁵ (or its acid addition salt) in a solvent such as DMF, DMA, NMP, dioxane, DMSO, or toluene, optionally in the presence of a base such as triethylamine, at a temperature between about 100° C. and 250° C. The reaction may be carried out under microwave irradiation.

In the case where one of the G groups is nitrogen, or where all of the G groups are carbon, compounds of formula (XXVI) can be converted to a mixture of the compounds of formulas (XXVII) and (XXVIII) by the reaction with an amine of structure HNR⁴R⁵ (or its acid addition salt) in a solvent such as DMF, DMA, dioxane, NMP, or toluene, in the presence of a suitable transition metal catalyst system. Suitable systems include the system consisting of copper(I) iodide, proline, and potassium carbonate (J. Org. Chem., 2005, 70, 5164), or the system consisting of a palladium species such as palladium(II) acetate, a chelating ligand such as 1,1′-BINAP, and a base such as cesium carbonate.

Compounds of Formula (XIII) may alternatively be prepared as shown in Scheme 9.

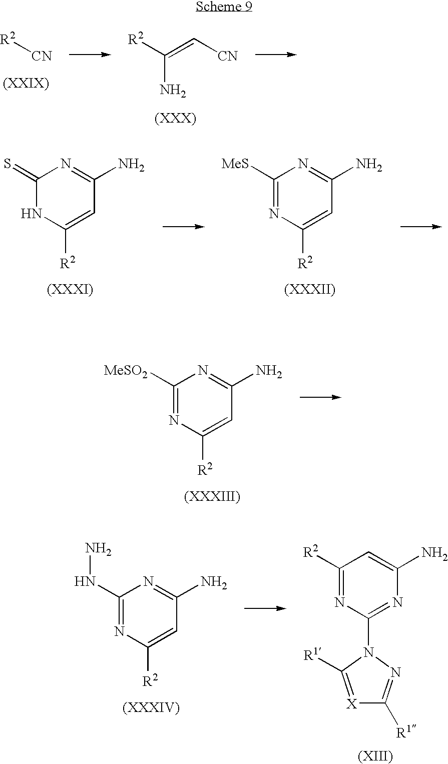

A nitrile of formula (XXIX) is reacted with acetonitrile in the presence of a suitable base such as sodium ethoxide or potassium tert-butoxide, in a suitable solvent such as toluene, at a temperature from about 0° C. to about 100° C., to provide an enaminonitrile of formula (XXX).

The compound of formula (XXX) is in turn converted to a thiopyrimidinone of formula (XXXI) by reaction with thiourea in the presence of a base such as sodium ethoxide or sodium methoxide, in a solvent such as ethanol, at a temperature from about 60° C. to about 100° C.

The methylthio compounds of formula (XXXII) are obtained from compounds of formula (XXXI) by reaction with iodomethane or dimethylsulfate, in aqueous sodium or potassium hydroxide, at a temperature of about 0° C. to about 50° C.

Methylsulfone compounds of formula (XXXIII) are obtained by oxidation of compounds of formula (XXXII) using a suitable oxidizing agent such as OXONE®, hydrogen peroxide, potassium permanganate, a peracid, or sodium perborate. In some cases, oxidation of the R² group can occur concomitantly. Such oxidations include oxidation of amino or pyridine groups to the corresponding N-oxide, and can be reversed in the subsequent hydrazine displacement step.

The sulfone compounds of formula (XXXIII) can be converted to hydrazine derivatives of formula (XXXIV) by reaction with hydrazine in the presence of a solvent such as ethanol or NMP at a temperature of room temperature to 80° C., optionally in the presence of a base such as pyridine. In cases in which oxidation of the R² group had occurred in the previous step, the hydrazine treatment serves to reduce the oxidized R² functionality, restoring the original R² functionality present in compounds of formula (XXXII).

Compounds of formula (XXXIV) are converted to compounds of formula (XIII) by treatment with the appropriate dicarbonyl compound, in a solvent such as ethanol or dioxane, at a temperature of between room temperature and about 100° C.

Compounds of formula (IX), in which R¹ is connected to the pyrimidine ring via a carbon atom, may alternatively be obtained by reaction of an amidine salt of formula (II) with an enaminonitrile of formula (XXX) as shown in Scheme 10.

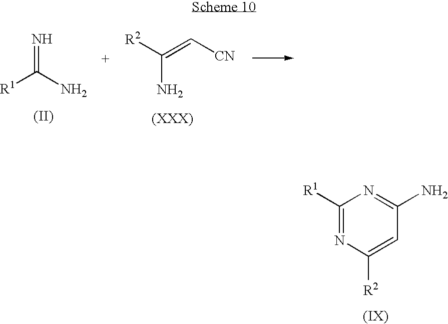

The reaction is performed in a solvent such as ethanol or dioxane, in the presence of a base such as potassium tert-butoxide or sodium ethoxide, at a temperature of between about 60° C. and 200° C., and may be performed with microwave irradiation.

Compounds of formula (XXXVI) are special cases of compounds of formula (I) and may be prepared as shown in Scheme 11.

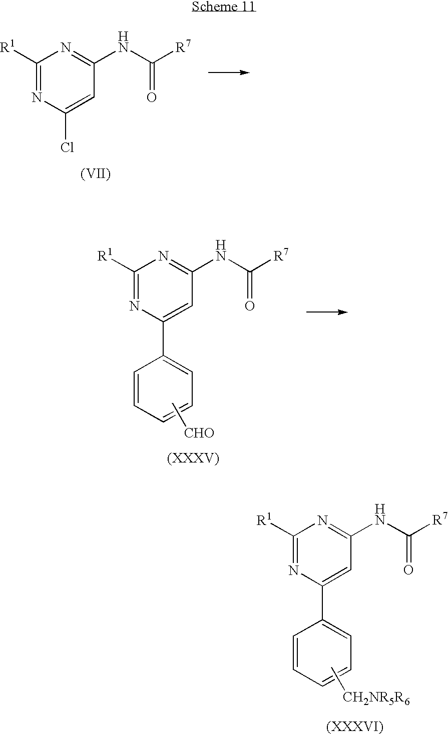

Compounds of formula (VII) are reacted with a compound of formula R²-M, wherein R² is phenyl substituted at a minimum with a formyl group, to yield the compounds of formula (XXXV). M is a suitable metal such as boron substituted with additional ligands as appropriate. Suitable ligated metallic groups include boronic acid or ester. The reaction is carried out in a solvent such as dioxane, tetrahydrofuran, dimethylformamide, dimethylacetamide or toluene, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C. Benzaldehydes of formula (XXXV) are converted to compounds of formula (XXXVI) by treatment with a primary or secondary amine in the presence of a suitable reducing agent such as sodium triacetoxyborohydride, sodium cyanoborohydride, or pyridine-borane complex. Reductions with sodium triacetoxyborohydride are performed in an inert solvent such as dichloroethane, tetrahydrofuran, or dichloromethane, in the presence of an acid such as acetic acid, at a temperature of about 0° C. to room temperature. Reductions with sodium cyanoborohydride and pyridine-borane complex are performed in an alcoholic solvent such as methanol, at a temperature of about 0° C. to room temperature.

Compounds of formula (XXXIX) are special cases of compounds of formula (I) and may be prepared as shown in Scheme 12.

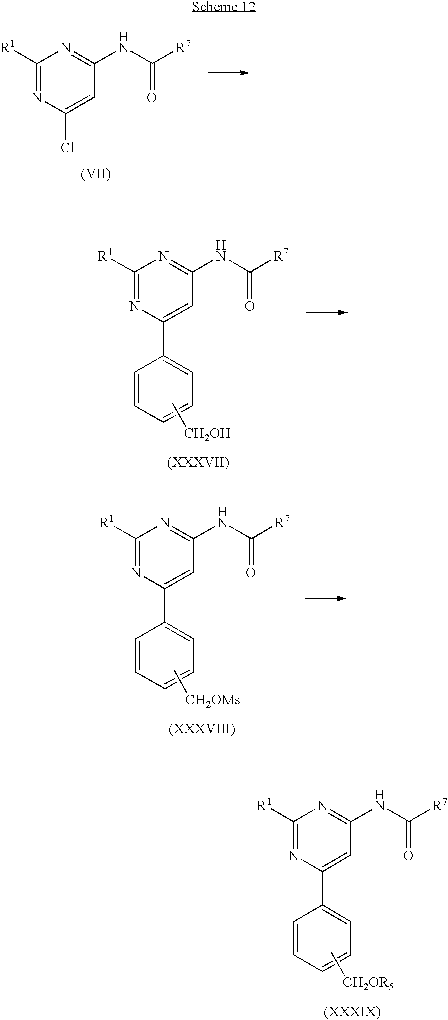

Compounds of formula (VII) are reacted with a compound of formula R²-M, wherein R² is phenyl substituted at a minimum with a hydroxymethyl group, to yield the compounds of formula (XXXVII). M is a suitable metal such as boron substituted with additional ligands as appropriate. Suitable ligated metallic groups include boronic acid or ester. The reaction is carried out in a solvent such as dioxane, tetrahydrofuran, dimethylformamide, dimethylacetamide or toluene, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C. Hydroxymethyl compounds of formula (XXXVII) can also be prepared by reduction of benzaldehyde compounds of formula (XXXV) using sodium borohydride in an alcoholic solvent, such as methanol or ethanol, at a temperature of about 0° C. to room temperature. Hydroxymethyl compounds of formula (XXXVII) are converted to the corresponding methanesulfonate esters of formula (XXXVIII) by treatment with methanesulfonyl chloride at a temperature of about 0° C. to room temperature, in the presence of an amine base such as triethylamine or diisopropylethylamine, in an inert solvent such as dichloromethane or tetrahydrofuran. The methanesulfonate esters of formula (XXXVIII) are converted to compounds of formula (XXXIX) by treatment with an alcohol R⁵OH and a base such as sodium hydride, in a suitable solvent such as dimethylformamide or dioxane, optionally in the presence of a nucleophilic catalyst such as sodium iodide, at a temperature of about 0° C. to 100° C.

Compounds of formula (XXXXIII) are special cases of compounds of formula (I) and may be prepared as shown in Scheme 13.

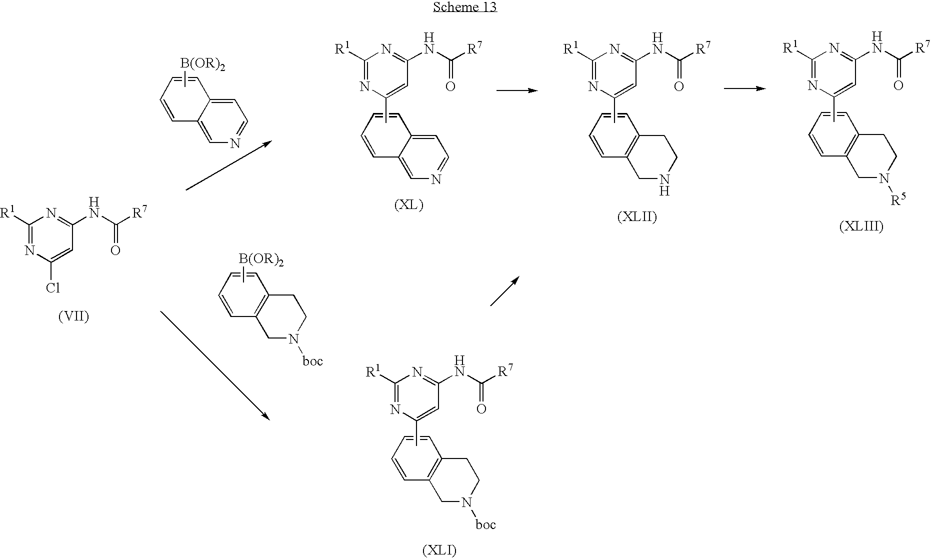

Suzuki coupling of a compound of formula (VII) with an isoquinolinyl boronic acid or ester produces an isoquinoline compound of formula (XL). Similarly, Suzuki coupling of a compound of formula (VII) with a boc-protected tetrahydroisoquinolinyl boronic acid or ester produces a boc-protected tetrahydroisoquinoline compound of formula (XLI). The reactions are carried out in a solvent such as dioxane, tetrahydrofuran, dimethylformamide, dimethylacetamide or toluene, in the presence of a base, such as sodium carbonate, potassium carbonate or cesium carbonate, in the presence of a suitable transition metal catalyst such as tetrakis(triphenylphosphine)palladium(0) at a temperature from 50° C. to 140° C. The isoquinolinyl compounds of formula (XL) are in turn reduced to the tetrahydroisoquinolinyl compounds of formula (XLII) using catalytic hydrogenation over a transition metal catalyst such as palladium on charcoal, in a solvent such as ethanol, acetic acid, or a mixture of the two, at a pressure of about 1 to 10 atmospheres. Boc-protected tetrahydroisoquinolinyl compounds of formula (XLI) also furnish tetrahydroisoquinolinyl compounds of formula (XLII) through BOC-deprotection using trifluoroacetic acid in dichloromethane or hydrogen chloride in dioxane, at a temperature of about 0° C. to room temperature. The compounds of formula (XLII) are converted to compounds of formula (XLIII) by treatment with an aldehyde in the presence of a suitable reducing agent such as sodium triacetoxyborohydride, sodium cyanoborohydride, or pyridine-borane complex. Reductions with sodium triacetoxyborohydride are performed in an inert solvent such as dichloroethane, tetrahydrofuran, or dichloromethane, in the presence of an acid such as acetic acid, at a temperature of about 0° C. to room temperature. Reductions with sodium cyanoborohydride and pyridine-borane complex are performed in an alcoholic solvent such as methanol, at a temperature of about 0° C. to room temperature. When the reduction of isoquinolines of formula (XL) to tetrahydroisoquinolines of formula (XLII) is performed in an alcoholic solvent, a by-product of the reaction is the N-alkylated product (XLIII), in which R⁵ is the alkyl group donated by the alcohol (e.g. ethyl for a reaction performed in ethanol).

When the defined groups R¹ to R⁷ are susceptible to chemical reaction under the conditions of the hereinbefore described processes or are incompatible with said processes, conventional protecting groups may be used in accordance with standard practice, for example see T. W. Greene and P. G. M. Wuts in ‘Protective Groups in Organic Chemistry’, 3^{rd} Edition, John Wiley & Sons (1999). It may be that deprotection will form the last step in the synthesis of compounds of formula (I).

### Pharmacological Activity

### Adenosine A_{2A} Receptor Binding Assays

### Receptor Cloning

The coding sequence of the human A_{2A} receptor was amplified from a human brain cDNA library by the polymerase chain reaction. The amplicon was cloned into the pcDNA5/FRT/V5-His-TOPO expression vector (Invitrogen) and sequence confirmed using an ABI 3100 automated sequencer (Applied Biosystems). The expression construct was transfected into Flp-In HEK cells (Invitrogen) using Lipofectamine 2000 (Invitrogen). Cells stably expressing the human A_{2A} receptor were selected using 1 mg/ml hygromycin in complete DMEM.

### Membrane Preparation

Crude membranes were prepared from Flp-In HEK cells transfected with the human A_{2A} receptor by resuspending cells in lysis buffer (50 mM Tris-HCl pH 7.4, 5 mM EDTA, 10 mM MgCl₂) and disrupting under N₂ at a pressure of 900 psi (Parr Cell disruption bomb, cat.4639) for 30 mM on ice followed by differential centrifugation. The resulting crude membrane pellet was resuspended in assay buffer (50 mM Tris HCl pH 7.4, 1 mM EDTA, 10 mM MgCl₂). Membrane protein concentration was determined by Bradford assay and aliquots were stored at −80° C.

### Binding Assay

An aliquot of membranes (5-10 μg of protein) was pre-incubated for 30 min at RT in the presence of 10 μg/ml Adenosine Deaminase (Type IV Calf Spleen, Sigma). Membranes were then incubated for 90 min with 1.0 nM [³H]-ZM 241385 (27.40 Ci/mmol Tocris R1036) in the presence of varying concentrations of competing ligand. Non-specific binding was determined in the presence of excess (1 μM) of CGS15943. Bound and free ligand were separated by rapid vacuum filtration using a Packard 96-well cell harvester onto UniFilter GF/C filter plates (PerkinElmer) that had been pretreated with 0.5% polyethyleneimine. The filter plates were than washed 3×200 μl with 50 mM Tris HCl, 50 mM NaCl pH 7.4. Bound radioligand was determined by scintillation counting using a TopCount-NXT (Packard). Binding data was analyzed by nonlinear, least-squares curve fitting algorithms using GraphPad Prism (GraphPad Software, Inc. San Diego, Calif.) or ActivityBase (IDBS, Guildford, Surrey, UK). Kᵢ values were calculated from IC₅₀ values using the Cheng-Prusoff equation (Cheng, Y, Prusoff, W. H. Biochem. Pharm. 22:3099-3108, 1973.).

For A_{2A} membrane assay:
ZM241385 measured Kd=0.3±0.2 nM; Bₘₐₓ=33±8 pmol/mg by Scatchard Analysis
   Binding Ki=0.25±0.04 nM.

With reference to A_{2A} receptor binding affinities, A_{2A} receptor antagonists of this invention may have a Ki of less than 10 μM. In one embodiment of this invention, a A_{2A} receptor antagonist has a Ki of less than 1

The compounds of the invention may be useful in the treatment or prevention of diseases that are susceptible to improvement by treatment with an antagonist of an adenosine receptor, in particular those susceptible to improvement by treatment with and antagonist of the A_{2A} adenosine receptor. Such diseases include, for example ischemia, supraventricular arrhythmias, acute renal failure, myocardial reperfusion injury, allergic reactions including but not limited to rhinitis, urticaria, scieroderm arthritis, other autoimmune diseases, inflammatory bowel diseases, asthma, diabetes mellitus, obesity, Parkinson disease, Huntington's disease, dystonias such as restless leg syndrome, dyskinesias such as those caused by prolonged use of neuroleptic and dopaminergic drugs, and sleep disorders.

Accordingly, the compounds of the invention and pharmaceutically acceptable salts thereof, and pharmaceutical compositions comprising such compound and/or salts thereof, may be used in a method of treatment of disorders of the human body which comprises administering to a subject requiring such treatment an effective amount of a compound of the invention or a pharmaceutically acceptable salt thereof.

The present invention also provides pharmaceutical compositions which comprise, as an active ingredient, at least a compound of formula (I) or a pharmaceutically acceptable salt thereof in association with a pharmaceutically acceptable excipient such as a carrier or diluent. The active ingredient may comprise 0.001% to 99% by weight, preferably 0.01% to 90% by weight of the composition depending upon the nature of the formulation and whether further dilution is to be made prior to application. Preferably the compositions are made up in a form suitable for oral, topical, nasal, rectal, percutaneous or injectable administration.

The pharmaceutically acceptable excipients which are admixed with the active compound, or salts of such compound, to form the compositions of this invention are well-known per se and the actual excipients used depend inter alia on the intended method of administering the compositions.

Compositions of this invention are preferably adapted for injectable and per os administration. In this case, the compositions for oral administration may take the form of tablets, retard tablets, sublingual tablets, capsules, inhalation aerosols, inhalation solutions, dry powder inhalation, or liquid preparations, such as mixtures, elixirs, syrups or suspensions, all containing the compound of the invention; such preparations may be made by methods well-known in the art.

The diluents which may be used in the preparation of the compositions include those liquid and solid diluents which are compatible with the active ingredient, together with colouring or flavouring agents, if desired. Tablets or capsules may conveniently contain between 2 and 500 mg of active ingredient or the equivalent amount of a salt thereof.

The liquid composition adapted for oral use may be in the form of solutions or suspensions. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent.

Compositions for parenteral injection may be prepared from soluble salts, which may or may not be freeze-dried and which may be dissolved in pyrogen free aqueous media or other appropriate parenteral injection fluid.

Effective doses are normally in the range of 2-2000 mg of active ingredient per day. Daily dosage may be administered in one or more treatments, preferably from 1 to 4 treatments, per day.

The present invention will be further illustrated by the following examples. The examples are given by way of illustration only and are not to be construed as a limiting.

Reagents, starting materials, and solvents were purchased from commercial suppliers and used as received. Concentration refers to evaporation under vacuum using a Büchi rotatory evaporator. Reaction products were purified, when necessary, by flash chromatography on silica gel (40-63 μm) with the solvent system indicated. Spectroscopic data were recorded on a Varian Mercury 300 MHz Spectrometer and a Bruker Avance 500 MHz spectrometer.

### Analytical HPLC-MS Method 1

Platform: Agilent 1100 series: equipped with an auto-sampler, an UV detector (220 nM and 254 nM), a MS detector (APCI);

HPLC column: Phenomenex Synergi-Max RP, 2.0×50 mm column;

HPLC gradient: 1.0 mL/minute, from 5% acetonitrile in water to 95% acetonitrile in water in 13.5 minutes, maintaining 95% for 2 minute. Both acetonitrile and water have 0.025% TFA.

### Analytical HPLC-MS Method 2

Platform: Dionex: equipped with an autosampler, an UV detector (220 nM and 254 nM), a MS detector (APCI);

HPLC column: Phenomenex CX18 4.6×150 mm;

HPLC gradient: 95% 0.04% NH₄OH/H2O to 90% 0.04% NH₄OH/ACN over 9.86 min, 12.30 min run

### Analytical HPLC-MS Method 3

Platform: Agilent 1100 series: equipped with an auto-sampler, an UV detector (220 nM and 254 nM), a MS detector (APCI);

HPLC column: Phenomenex Synergi-Max RP, 2.0×50 mm column;

HPLC gradient: 1.0 mL/minute, from 10% acetonitrile in water to 90% acetonitrile in water in 2.5 minutes, maintaining 90% for 1 minute. Both acetonitrile and water have 0.025% TFA.

### Analytical HPLC-MS Method 4

Platform: Agilent 1100 series: equipped with an auto-sampler, an UV detector (230 nM and 254 nM), a MS detector (APCI);

HPLC column: Phenomenex Synergi-Max RP, 2.0×50 mm column;

HPLC gradient: Solvent C is 6 mM Ammonium Formate in water, solvent D is 25% Acetonitrile in Methanol. The gradient runs from 5% D (95% C) to 95% D (5% C) in 6.43 min with a 1.02 min hold at 95% D followed by a return and hold at 5% D for 1.52 min.

### Intermediate 1: 6-Chloro-2-(3,5-dimethyl-pyrazol-1-yl)-pyrimidin-4-ylamine

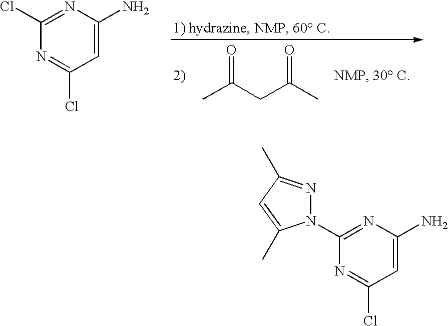

4-amino-2,6-dichloropyrimidine (40.0 g, 0.24 mol) was dissolved in NMP (200 ml). The slurry was heated to 60° C. and anhydrous hydrazine (19.1 ml, 0.61 mol) was added slowly. After 1.5 hours, the addition was complete. The reaction was cooled to rt and 2,4-pentanedione (63 ml, 0.61 mol) was added slowly, keeping the reaction temperature below 50° C. After one hour, the reaction was heated again at 50° C. then ethanol (200 ml) was added, followed by water (400 ml). Once the water addition was complete, the reaction mixture was cooled to rt, filtered on paper. The cake was washed with alcohol/water (3×200 mL) and dried under vacuum at 60° C. overnight. The recovered tan solid was a mixture of the desired regioisomer (43 g) and the 4-dimethylpyrazole regioisomer. The product was recrystallized from hot THF/1-PrOAc to give a white solid (yield 66%). LCMS (Method 3) m/z 223.9 [MH+], Tr=1.97 min.

### Intermediate 2: N-[6-Chloro-2-(3,5-dimethyl-pyrazol-1-yl)-pyrimidin-4-yl]-acetamide

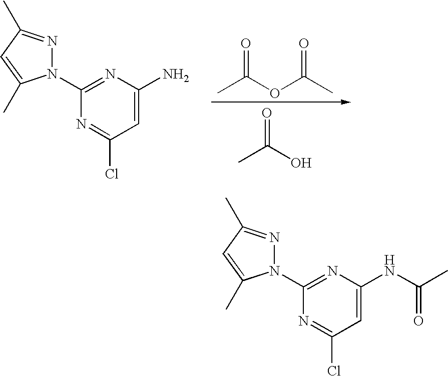

6-Chloro-2-(3,5-dimethyl-pyrazol-1-yl)-pyrimidin-4-ylamine (40 g, 0.18 mol) was dissolved in acetic acid (200 ml) and stirred at rt. Acetic anhydride (80 ml, 0.8 mol) was added and the mixture was heated at 90° C. overnight. Once the reaction was complete, it was cooled to room temperature and 16 ml of water was added over 30 minutes. The mixture was then filtered through filter paper and the cake was washed with water (4×75 mL). The solid was dried in a vacuum oven at 50° C. overnight. The product obtained (acetic acid solvate, 48.2 g, 83% yield) was an off-white crystalline solid. LCMS (Method 3) m/z 265.9 [MH+], Tr=2.11 min.

### Intermediate 3: N-[6-(6-chloropyridin-2-yl)-2-(3,5-dimethyl-pyrazol-1-yl)-pyrimidin-4-yl]-acetamide

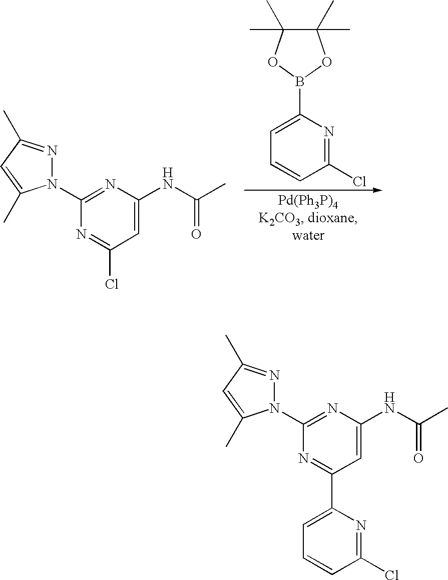

A mixture of Intermediate 2 (1.8 g, 6.8 mmol), 6-chloropyridin-2-boronic acid pinacol ester (2.27 g, 9.5 mmol), potassium carbonate (1.88 g, 13.6 mmol), dioxane (35 ml) and water (3.5 ml) was sparged with nitrogen gas for 5 minutes. Tetrakis(triphenylphosphine)palladium(0) (785 mg, 0.68 mmol) was added, the mixture was sparged with nitrogen gas for an additional 10 minutes, then the vessel was sealed and heated with stirring at 90° C. for 16 h. Water (50 ml) was added and the resulting solid was collected, rinsed with water and ethyl acetate, then dried under vacuum to provide the title compound (1.53 g, 4.5 mmol, 66%) as a pinkish solid. LCMS (Method 3) m/z 342.8, 344.8 [MH+], Tr=2.65 min.

### Intermediate 4: N-[6-(2-chloropyridin-4-yl)-2-(3,5-dimethyl-pyrazol-1-yl)-pyrimidin-4-yl]-acetamide

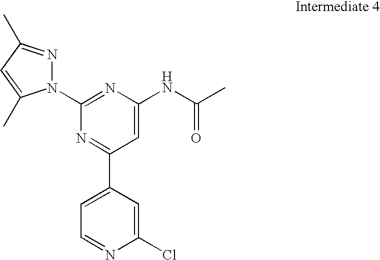

Intermediate 4 was prepared according to the procedures described in Intermediate 3, except that 2-chloropyridin-4-boronic acid was used instead of 6-chloropyridin-2-boronic acid pinacol ester. (yellow solid, 40% yield) LCMS (Method 3) m/z 342.8 [MH+], Tr=2.54 min.

### Intermediate 5: N-[6-(5-bromopyridin-3-yl)-2-(3,5-dimethyl-pyrazol-1-yl)-pyrimidin-4-yl]-acetamide

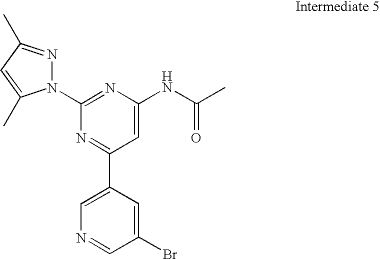

Intermediate 5 was prepared according to the procedures described in Intermediate 3, except that 3-bromopyridine-5-boronic acid was used instead of 6-chloropyridin-2-boronic acid pinacol ester. (white solid, 11% yield following silica gel chromatography, 0-10% methanol in dichloromethane as eluant) LCMS (Method 1) m/z 386.7, 388.7 [MH+], Tr=6.01 min.

### Compounds 1-1 and 1-2: N-[2-(3,5-Dimethyl-pyrazol-1-yl)-6-(2-pyrrolidin-1-yl-pyridin-4-yl)-pyrimidin-4-yl]-acetamide and N-[2-(3,5-Dimethyl-pyrazol-1-yl)-6-(2-pyrrolidin-1-yl-pyridin-4-yl)-pyrimidin-4-yl]-amine

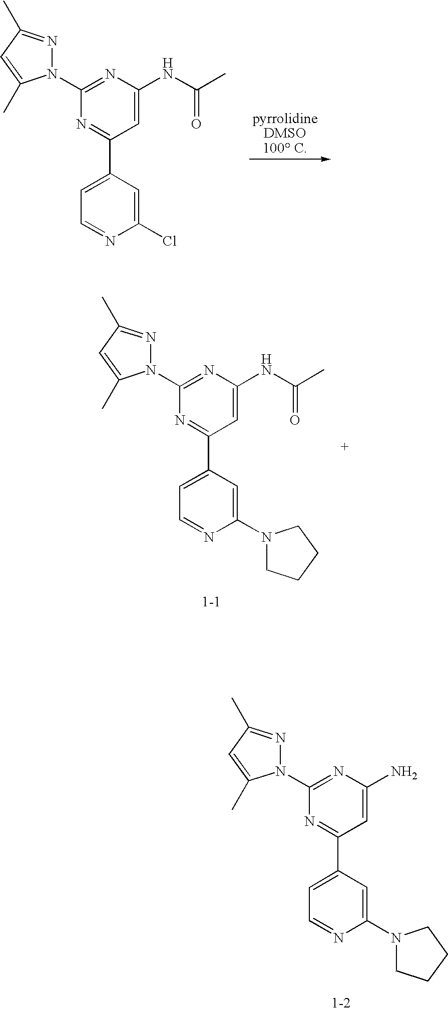

A mixture of Intermediate 4 (200 mg, 0.58 mmol), pyrrolidine (0.2 ml, 2.4 mmol), and DMSO (2 ml) was heated in a sealed tube with stirring at 100° C. for 3 hours. The reaction mixture was diluted with methanol, filtered, and purified by HPLC using 5-65% acetonitrile in water (0.05% TFA) to give the title compounds as TFA salts.

### Compounds 1-3 and 1-4: N-[2-(3,5-Dimethyl-pyrazol-1-yl)-6-(4-methoxy-3,4,5,6-tetrahydro-2H-[1,2′]bipyridinyl-6′-yl)-pyrimidin-4-yl]-acetamide and 2-(3,5-Dimethyl-pyrazol-1-yl)-6-(4-methoxy-3,4,5,6-tetrahydro-2H-[1,2′]bipyridinyl-6′-yl)-pyrimidin-4-ylamine

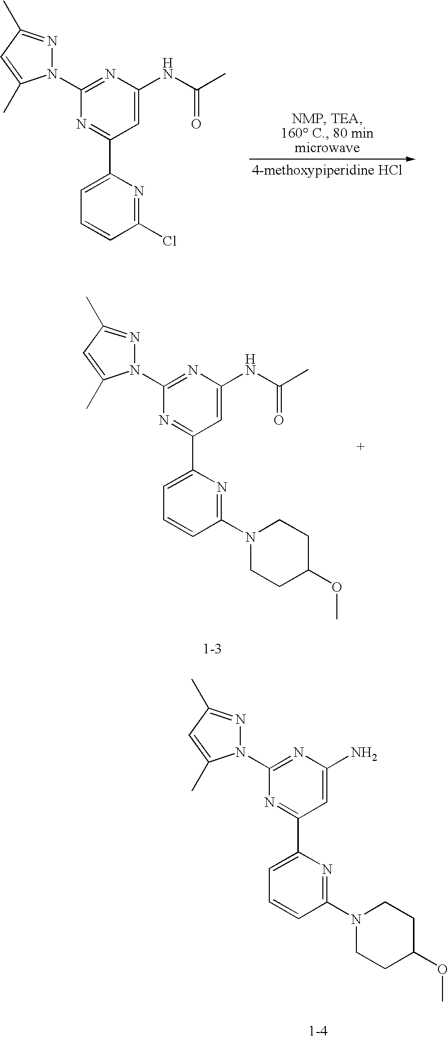

A mixture of Intermediate 3 (60 mg, 0.17 mmol), 4-methoxypiperidine hydrochloride (128 mg, 0.84 mmol), triethylamine (0.12 ml, 0.86 mmol), and NMP (0.45 ml) was heated in a microwave reactor at 160° C. for 80 min. The reaction mixture was diluted with methanol, filtered, and purified by HPLC using 15-75% acetonitrile in water (0.05% TFA) to provide Compounds 1-3 and 1-4 as TFA salts.

### Compounds 1-5 and 1-6: N-(2-(3,5-Dimethyl-pyrazol-1-yl)-6-{6-[(2-methoxy-ethyl)-methyl-amino]pyridin-2-yl}-pyrimidin-4-yl)acetamide and 2-(3,5-Dimethyl-pyrazol-1-yl)-6-{6-[(2-methoxyethyl)-methylamino]-pyridin-2-yl}-pyrimidin-4-ylamine

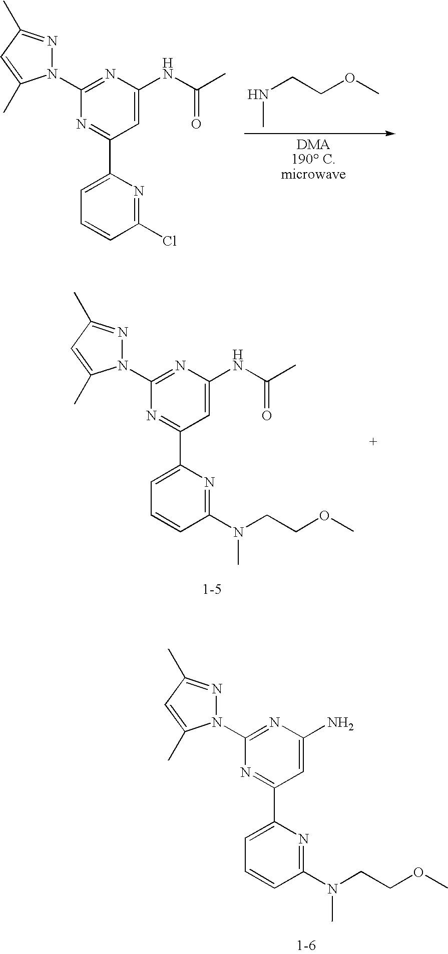

A mixture of Intermediate 3 (250 mg, 0.73 mmol), N-(2-methoxyethyl)methylamine (0.5 g, 5.6 mmol), and DMA (1.5 ml) was heated in a microwave reactor at 190° C. for 20 min. A second replicate of this procedure was run (500 mg total input of Intermediate 3). The two reaction mixtures were combined, aqueous sodium bicarbonate was added, and the mixture was extracted with dichloromethane. The combined organic extracts were dried over sodium sulfate, filtered, and concentrated. The residue was purified by preparative thin layer (9:1 dichloromethane/methanol with 1% ammonium hydroxide as eluant) to provide Compound 1-5 (70 mg 12%), along with Compound 1-6 (4 mg).

The compounds of Table 1 were prepared by reacting the appropriate intermediate with the appropriate amine representing the R² substituent according to the methods described above for preparation of Compounds 1-1, 1-2, 1-3, 1-4, 1-5, and 1-6. In some cases, a TFA salt product was converted to the free base by extraction from aqueous sodium bicarbonate, and in some cases the resulting free base was converted to a hydrochloride salt by treatment with a solution of hydrogen chloride in ether.

TABLE 1

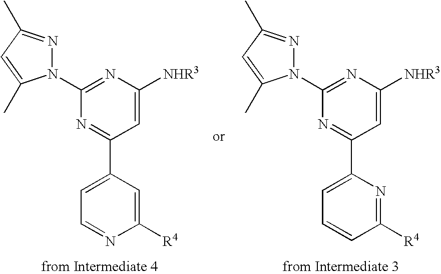

| | | | | | | Reten | | |
|---|---|---|---|---|---|---|---|---|
| Cmpd | Inter- | | | | MS | time | HPLC | |
| No. | Mediate | R⁴ | R³ | MW | ION | (min) | Method | Salt |
| 1-1 | 4 | Pyrrolidin-1-yl | Ac | 377.4 | 378 | 3.96 | 1 | TFA |
| 1-2 | 4 | Pyrrolidin-1-yl | H | 335.4 | 336 | 3.7 | 1 | TFA |
| 1-3 | 3 | 4-methoxy- | Ac | 421.5 | 421.9 | 6.05 | 1 | TFA |
| | | piperidin-1-yl | | | | | | |
| 1-4 | 3 | 3-methoxy- | H | 379.5 | 379.9 | 5.18 | 2 | TFA |
| | | piperidin-1-yl | | | | | | |
| 1-5 | 3 | N-methyl-N-(2- | Ac | 395.5 | 395.9 | 4.96 | 1 | Free |
| | | methoxyethyl)- | | | | | | Base |
| | | amino | | | | | | |
| 1-6 | 3 | N-methyl-N-(2- | H | 353.4 | 354 | 4.58 | 1 | Free |
| | | methoxyethyl)- | | | | | | Base |
| | | amino | | | | | | |
| 1-7 | 3 | morpholin-4-yl | Ac | 393.4 | 393.8 | 6.08 | 1 | Free |
| | | | | | | | | Base |
| 1-8 | 3 | 4-methyl- | Ac | 406.5 | 406.9 | 4.26 | 1 | Free |
| | | piperazin-1-yl | | | | | | Base |
| 1-9 | 3 | 4-hydroxy- | Ac | 407.5 | 407.9 | 4.96 | 1 | TFA |
| | | piperidin-1-yl | | | | | | |
| 1-10 | 3 | [1,4]oxazepan- | Ac | 407.5 | 407.9 | 5.92 | 1 | TFA |
| | | 4-yl | | | | | | |
| 1-11 | 3 | (R)-2- | Ac | 421.5 | 421.9 | 4.73 | 1 | Free |
| | | methoxymethyl- | | | | | | Base |
| | | pyrrolidin-1-yl | | | | | | |
| 1-12 | 3 | N-ethyl-N- | Ac | 393.5 | 393.9 | 6.58 | 1 | TFA |
| | | propylamino | | | | | | |
| 1-13 | 4 | morpholin-4-yl | Ac | 393.4 | 393.9 | 3.91 | 1 | Free |
| | | | | | | | | Base |
| 1-14 | 3 | N-ethyl-N-(2- | Ac | 409.5 | 409.9 | 5.3 | 1 | TFA |
| | | methoxyethyl)- | | | | | | |
| | | amino | | | | | | |
| 1-15 | 3 | piperidin-1-yl | Ac | 391.5 | 391.9 | 6.23 | 1 | TFA |
| 1-16 | 3 | N,N- | Ac | 351.4 | 352 | 4.58 | 1 | Free |
| | | dimethylamino | | | | | | Base |
| 1-17 | 3 | 4-methoxy- | Ac | 393.4 | 394 | 4.67 | 1 | TFA |
| | | azetidin-1-yl | | | | | | |
| 1-18 | 4 | 4-methyl- | Ac | 406.5 | 409.9 | 4.05 | 1 | TFA |
| | | piperazin-1-yl | | | | | | |
| 1-19 | 3 | pyrrolidin-1-yl | H | 335.4 | 336 | 4.36 | 1 | HCl |
| 1-20 | 3 | morpholin-4-yl | H | 351.4 | 351.9 | 4.89 | 1 | TFA |
| 1-21 | 3 | 4-methyl- | H | 364.5 | 364.9 | 3.5 | 1 | TFA |
| | | piperazin-1-yl | | | | | | |
| 1-22 | 4 | morpholin-4-yl | H | 351.4 | 351.9 | 3.46 | 1 | HCl |
| 1-23 | 3 | 4-hydroxy- | H | 365.4 | 365.9 | 4.22 | 1 | TFA |
| | | piperidin-1-yl | | | | | | |
| 1-24 | 3 | (R)-3-hydroxy- | H | 351.4 | 351.9 | 3.53 | 1 | TFA |
| | | pyrrolidin-1-yl | | | | | | |
| 1-25 | 3 | (R)-2- | H | 379.5 | 379.9 | 4.53 | 1 | Free |
| | | methoxymethyl- | | | | | | Base |
| | | pyrrolidin-1-yl | | | | | | |
| 1-26 | 3 | 4-methoxy- | H | 351.4 | 352 | 4.31 | 1 | TFA |
| | | azetidin-1-yl | | | | | | |
| 1-27 | 3 | N,N- | H | 309.4 | 309.9 | 4.3 | 1 | Free |
| | | dimethylamino | | | | | | Base |
| 1-28 | 3 | pyrrolidin-1-yl | Ac | 377.4 | 377.9 | 4.43 | 1 | Free |
| | | | | | | | | Base |

### Compounds 2-1 and 2-2: N-[2-(3,5-Dimethyl-pyrazol-1-yl)-6-(5-pyrrolidin-1-yl-pyridin-3-yl)-pyrimidin-4-yl]-acetamide and 2-(3,5-Dimethyl-pyrazol-1-yl)-6-(5-pyrrolidin-1-yl-pyridin-3-yl)-pyrimidin-4-ylamine

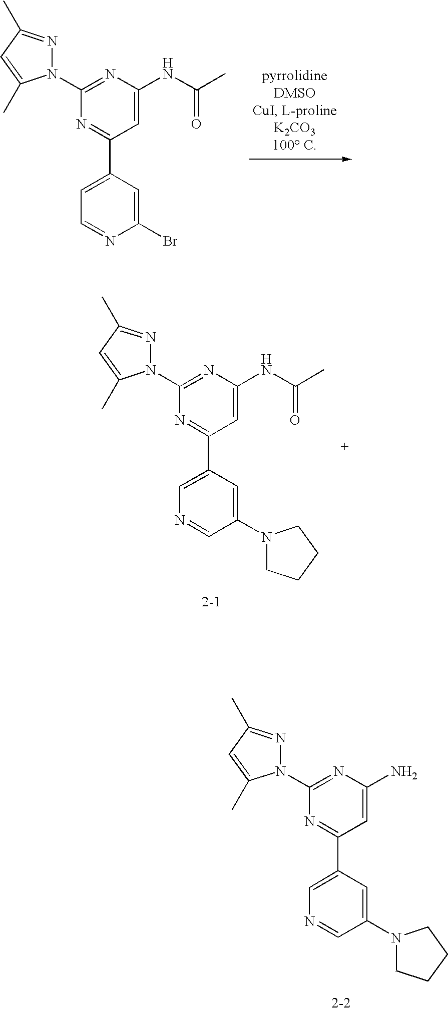

A mixture of Intermediate 5 (150 mg, 0.39 mmol), pyrrolidine (0.24 ml, 2.8 mmol), potassium carbonate (110 mg, 0.80 mmol), L-proline (50 mg, 0.43 mmol), copper(I) iodide (40 mg, 0.21 mmol), and DMSO (2 ml) was heated in a sealed tube with stirring at 100° C. for 6 hr. The cooled reaction mixture was diluted with methanol, filtered, and purified by HPLC using 5-65% acetonitrile in water (0.05% TFA) to give both title compounds as TFA salts. Compound 2-1 was converted to the free base by extraction.

### Compound 2-3: N-[2-(3,5-Dimethyl-pyrazol-1-yl)-6-(5-morpholin-4-yl-pyridin-3-yl)-pyrimidin-4-yl]-acetamide

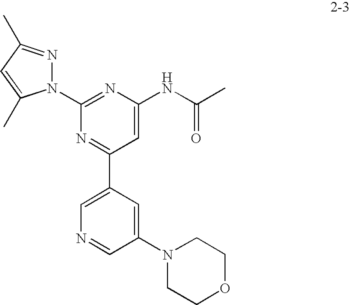

Compound 2-3 was prepared according to the procedures described in Compound 2-1, except that morpholine was used instead of pyrrolidine.

Characterization data for Compounds 2 are shown in Table 2:

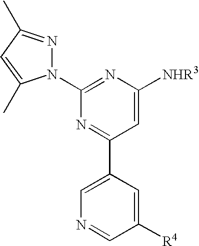

TABLE 2

| Cmpd | Inter- | | | | MS | Reten | HPLC | |
|---|---|---|---|---|---|---|---|---|
| No. | Mediate | R⁴ | R³ | MW | ION | time (min) | Method | Salt |
| 2-1 | 5 | pyrrolidin-1-yl | H | 335.41 | 336.0 | 4.11 | 1 | TFA |
| 2-2 | 5 | pyrrolidin-1-yl | Ac | 377.45 | 377.9 | 4.30 | 1 | Free |
| | | | | | | | | Base |
| 2-3 | 5 | morpholin-4-yl | Ac | 393.45 | 393.9 | 3.86 | 1 | Free |
| | | | | | | | | Base |

### Intermediate 6: Ethyl 6-(morpholin-4-yl)pyridine-2-carboxylate

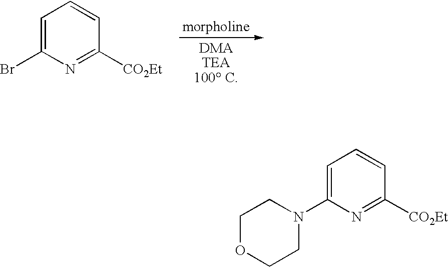

Morpholine (4.17 ml, 47.9 mmol, 1.05 eq) was added to a solution of ethyl 6-bromopyridine-2-carboxylate (10.5 g, 45.6 mmol, 1.0 eq) and triethylamine (9.5 ml, 68.4 mmol, 1.5 eq) in N,N-dimethylacetamide (20 ml). The mixture was heated at 100° C. in a sealed tube for 14.5 hr. Aqueous sodium bicarbonate was added and the mixture was extracted with ethyl acetate. The organic layer was dried over sodium sulfate, filtered, and concentrated to a brown oil. Chromatography (silica gel, 3:1 hexanes/EtOAc) gave the product as a yellow solid, 6.5 g. LCMS (Method 3) m/z 236.9 [MH+], Tr=2.35 min.

### Intermediate 7: 6-(Morpholin-4-yl)pyridine-2-carboxamide

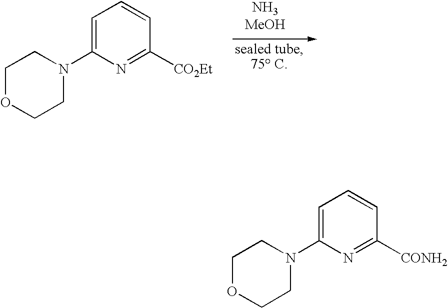

Intermediate 6 (6.3 g, 26.7 mmol) was dissolved in 7N ammonia in methanol (150 ml). The solution was heated with stirring in a sealed tube at 75° C. for 16 hr. The mixture was concentrated and the solid residue was co-evaporated with methanol, then ethyl acetate. The solid product was dried under vacuum to provide the title compound (5.46 g) as an off-white solid, which was used without further purification. LCMS (Method 3) m/z 207.9 [MH+], Tr=1.97 min.

### Intermediate 8: 6-(Morpholin-4-yl)pyridine-2-carbonitrile

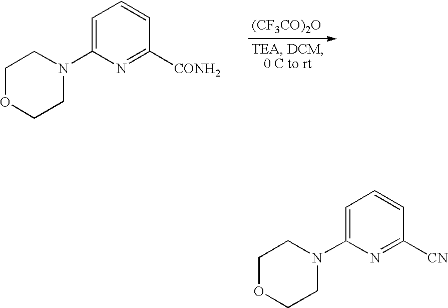

A suspension of the Intermediate 7 (5.4 g, 26 mmol) and triethylamine (7.3 ml, 52 mmol) in dichloromethane (100 ml) was cooled in an ice bath and treated dropwise with trifluoroacetic anhydride (4.0 ml, 29 mol). The mixture was allowed to stir at 0° C. for 15 min, then was allowed to warm to rt. After 30 min additional trifluoroacetic anhydride (0.5 ml, 3.6 mmol) was added and the mixture was stirred for another 30 min. Aqueous sodium bicarbonate solution was added and the mixture was extracted with dichloromethane. The organic layer was dried over sodium sulfate, filtered, and concentrated to a solid, which was triturated with 3:1 hexanes/ethyl acetate to provide the title compound (3.69 g) as a white solid. A second crop (0.66 g, slightly yellow solid) was obtained from the mother liquor. LCMS (Method 3) m/z 189.9 [MH+], Tr=2.38 min.

### Intermediate 9: 3-Amino-3-(6-morpholin-4-yl-pyridin-2-yl)-acrylonitrile

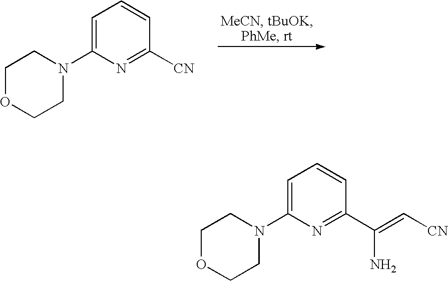

A mixture of Intermediate 8 (7.43 g, 39 mmol, 1.0 eq), anhydrous toluene (100 ml), and potassium t-butoxide (13.2 g, 118 mmol, 3.0 eq) was stirred without external heating or cooling while anhydrous acetonitrile (4.1 ml, 79 mmol, 2.0 eq) was added dropwise over 10 min. The thick yellow mixture was stirred for an additional 2 hr. Aqueous sodium bicarbonate solution was added (exothermic), then the mixture was extracted with ethyl acetate. The combined organic layers were dried over sodium sulfate, filtered, and concentrated to provide the title compound as a brown oil (9.6 g), which was used without purification. LCMS (Method 3) m/z 230.9 [MH+], Tr=2.38 min.

### Intermediate 10: 4-Amino-6-(6-morpholin-4-yl-pyridin-2-yl)-1H-pyrimidine-2-thione

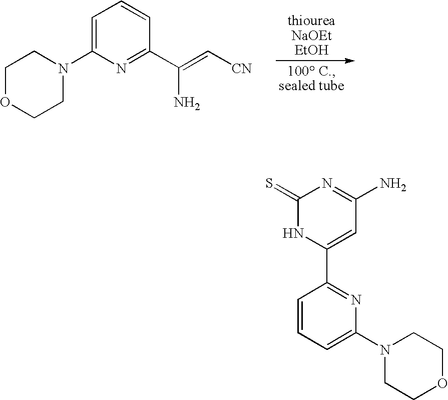

A suspension of Intermediate 9 (9.5 g, est 35 mmol) and thiourea (5.6 g, 74 mmol) in absolute ethanol (100 ml) was treated with sodium ethoxide solution (21 wt %, Aldrich, 27.5 ml, 74 mmol). The brown mixture was heated in a sealed tube at 100° C. for 49.5 hr. The cooled reaction mixture was poured onto 10% aqueous sodium dihydrogen phosphate solution (200 ml) chilled in an ice bath. The mixture was filtered. The filter cake was washed with water, then air dried briefly, to provide the title compound (34.6 g) as a tan gummy solid. This material was used without further purification. LCMS (Method 3) m/z 289.8 [MH+], Tr=1.97 min.

### Intermediate 11: 2-Methylsulfanyl-6-(6-morpholin-4-yl-pyridin-2-yl)-pyrimidin-4-ylamine

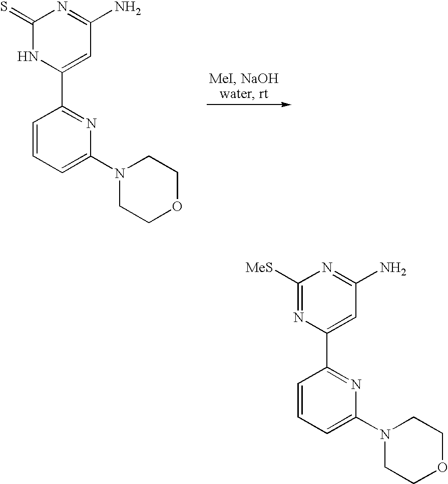

A suspension of Intermediate 10 (34.5 g, est 30 mmol) in 5% aqueous sodium hydroxide (100 ml, 125 mmol) was stirred at rt while iodomethane (1.86 ml, 30 mmol) was added in one portion. A light tan precipitate formed. After 45 min the mixture was treated with 10% aqueous sodium dihydrogen phosphate solution (final pH 9), then the solid was collected, washed with water, and air dried to provide the title compound (9.95 g) as a tan solid, still wet with water. LCMS (Method 3) m/z 303.9 [MH+], Tr=2.06 min.

### Intermediate 12: 2-Methanesulfonyl-6-[6-(4-oxy-morpholin-4-yl)-pyridin-2-yl]-pyrimidin-4-ylamine

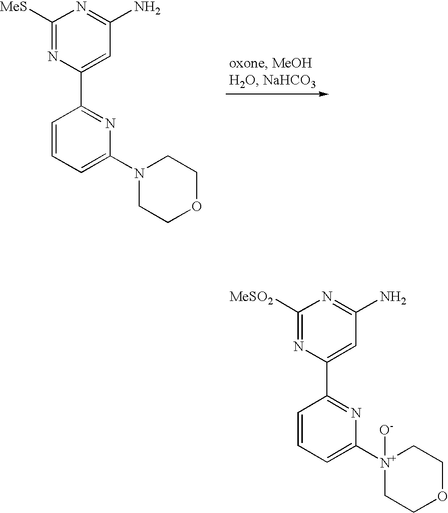

A suspension of Intermediate 11 (9.9 g, est 25 mmol) in methanol (200 ml) and water (50 ml) was cooled in an ice bath and treated dropwise with a solution of oxone (20.7 g) in water (100 ml) over 10 min. The temperature was kept below 20° C. during the addition. Saturated sodium bicarbonate solution (75 ml) was then added dropwise over 5 min (effervescence), and the ice bath was removed. After 1 hr, an additional portion of solid oxone (5.5 g) was added. After an additional 2 hr dichloromethane (200 ml) was added and the mixture was filtered. The filter cake was washed with water and air dried to provide the first product fraction as a tan solid (7.0 g). The two phases of the filtrate were separated. The aqueous phase was adjusted to pH>8 and was extracted with dichloromethane and ethyl acetate. The combined organic extracts were dried over sodium sulfate, filtered, and concentrated to provide the second product fraction (1.3 g) as a yellow solid. A slurry of product fractions one (6.8 g) and two (1.3 g) in ethanol was concentrated to dryness to provide the title compound (7.3 g) as a tan solid. LCMS (Method 3) m/z 351.9 [MH+], Tr=1.60 min.

### Intermediate 13: 2-Bromo-6-(morpholin-4-yl)pyridine

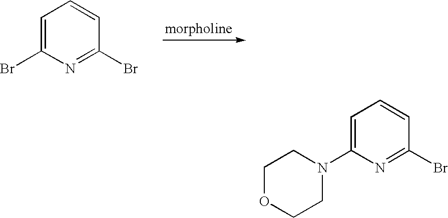

Morpholine (1.85 ml, 21.3 mmol) was added to a suspension of 2,6-dibromopyridine (5.0 g, 21.3 mmol) and cesium carbonate (6.88 g, 21.3 mmol) in DMF (15 ml). The mixture was heated with stirring at 120° C. for 2 hr. Water was added, the mixture was extracted with ether, and the combined extracts were washed with brine, dried over magnesium sulfate, filtered, and concentrated. The residue was chromatographed on silica gel using 5:1 hexanes/ethyl acetate as eluant to provide the title compound (4.33 g, 84%) as a white crystalline solid.

### Intermediate 14: 2-Bromo-6-(4-hydroxypiperidin-1-yl)pyridine

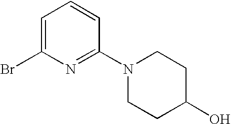

Intermediate 14 was prepared according to the procedures described in Intermediate 13, except that 4-hydroxy-piperidine was used instead of morpholine. LCMS (Method 3) m/z 256.8, 258.8 [MH+], Tr=2.11.

### Intermediate 15: 2-Bromo-6-(4-methoxypiperidin-1-yl)pyridine

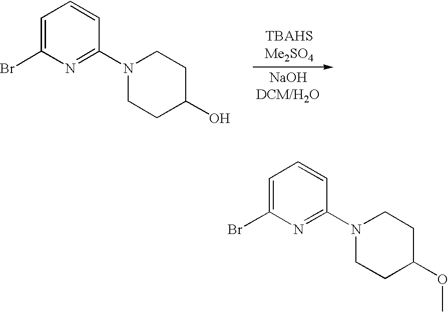

Tetrabutylammonium hydrogensulfate (6.93 g, 20.4 mmol) and dimethylsulfate (51.5 ml, 544 mmol) were added to a solution of Intermediate 14 (70 g, 272 mmol) in dichloromethane (300 ml). 50% aqueous sodium hydroxide (300 ml) was added and the mixture was stirred at rt for 16 hr, then was heated at reflux (50° C. bath) for 1 hr. Dichloromethane (400 ml) and water (1 L) were added to the cooled reaction mixture. The organic layer was washed twice with water and once with brine, then was dried over sodium sulfate, filtered, and concentrated to provide the title compound (68.6 g, 93%) as an amber oil, which was used without further purification. LCMS (Method 3) m/z 270.9, 272.9 [MH+], Tr=2.47.

### Intermediate 8 (alternate synthesis): 6-(Morpholin-4-yl)pyridine-2-carbonitrile

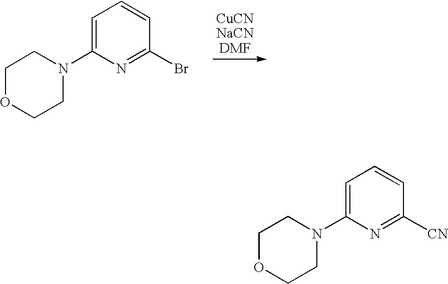

A suspension of copper (I) cyanide (24.3 g, 272 mmol) and sodium cyanide (8.9 g, 181 mmol) in DMF (160 ml) was heated at 150° C. until homogeneous. A solution of Intermediate 13 (44.0 g, 181 mmol) in DMF (40 ml) was added and the mixture was heated with stirring at 150° C. for 4 hr. The cooled reaction mixture was poured onto 1M potassium hydrogenphosphate solution (900 ml), then the mixture was stirred for 1 hr and filtered. The filter cake was rinsed with ether (500 ml), then was slurried with ethyl acetate (1 L), and the mixture filtered. The filtrate was dried over sodium sulfate, filtered, and concentrated to provide the title compound (28.3 g, 83%) as an off-white solid.

### Intermediate 16: 6-(4-methoxypiperidin-1-yl)pyridine-2-carbonitrile

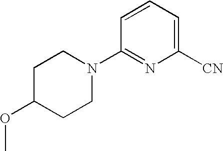

Intermediate 16 was prepared according to the procedures described in the alternative synthesis of Intermediate 8, except that Intermediate 15 was used instead of Intermediate 13. LCMS (Method 3) m/z 218.5 [MH+], Tr=2.23

### Intermediate 17: 5-Methyl-2-furancarboxamidine (HCl)

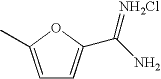

To a solution of sodium methoxide (5.55 mmol) in methanol (50 mL) was added 5-methyl-2-furonitrile (2.85 g, 26.6 mmol). The mixture was stirred at rt for 3 hr. To the resulting solution was slowly added ammonium chloride (1.57 g, 29.4 mmol) and the mixture was stirred at rt for 68 hours. The resulting suspension was filtered and the solvent removed under reduced pressure. The solid obtained was washed with ethyl ether (3×25 mL) to give 3.71 g (87% yield) of the title compound. ¹H NMR (300 MHz, DMSO-d6): δ 2.27 (s, 3H); 6.36 (d, J=3.6 Hz, 1H); 7.64 (d, J=3.6 Hz, 1H); 8.49 (bs, 4H).

### Compound 1-20 (alternate synthesis): 2-(3,5-Dimeth pyrazol-1-yl)-6-(6-morpholin-4-yl-pyridin-2-yl)-pyrimidin-4-ylamine

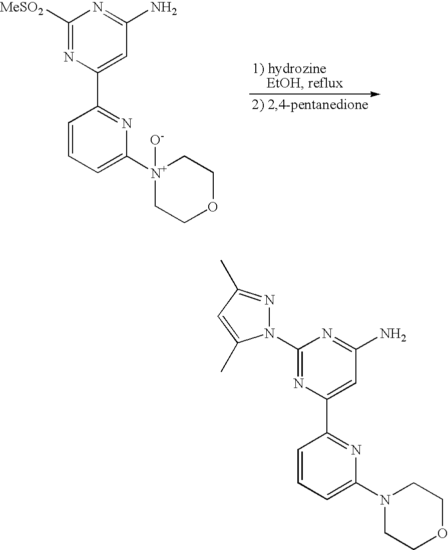

A suspension of Intermediate 12 (7.3 g, 19.4 mmol) in ethanol (200 ml) was treated with anhydrous hydrazine (6 ml, 189 mmol). The mixture was refluxed for 16 h. The mixture was allowed to cool to rt, then pentane-2,4-dione (20.5 ml, 200 mmol) was added dropwise over 30 min without external cooling. The mixture was heated at 70° C. for 1 h, then was allowed to cool and the solvent was removed. The resulting tan solid (30.5 g) was slurried with water (150 ml) at 40° C. for 15 min, then was allowed to cool. The cooled mixture was filtered and the filter cake was rinsed with water. The resulting filter cake was slurried with ethyl acetate and then concentrated to dryness to provide the product (5.9 g) as a tan solid, which contained the desired product in addition to 3,5-dimethylpyrazole (LCMS analysis). LCMS (Method 3) m/z 351.9 [MH+], Tr=2.27 min.

### Compound 3-1: 2-(3,5-Dimethyl-pyrazol-1-yl)-6-(4-morpholin-4-yl-pyridin-2-yl)-pyrimidin-4-ylamine

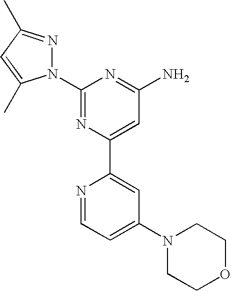

Compound 3-1 was prepared by the methods used for synthesis of Intermediates 6 through 12 and for the synthesis of Compound 1-20 (alternate method), starting with ethyl 4-bromopyridine-2-carboxylate in place of ethyl 6-bromopyridine-2-carboxylate. Purification by HPLC provided the title compound as a TFA salt. LCMS (Method 1) m/z 352.0 [MH+], Tr=3.85 min.

### Compound 1-3 (alternate synthesis): N-[2-(3,5-Dimethyl-pyrazol-1-yl)-6-(4-methoxy-3,4,5,6-tetrahydro-2H-[1,2′]bipyridinyl-6′-yl)-pyrimidin-4-yl]-acetamide

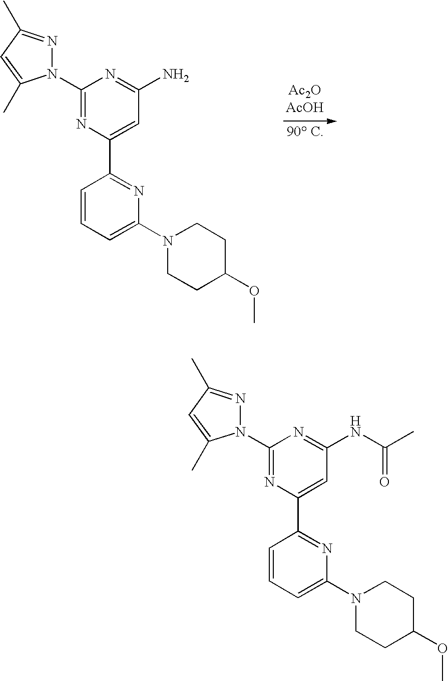

A mixture of Compound 1-4 (1.06 g, 2.8 mmol), acetic anhydride (0.50 ml, 5.3 mmol), and acetic acid (10 ml) was heated with stirring at 90° C. for 16 hr. Additional acetic anhydride (1.0 ml, 10.6 mmol) was added and the reaction was heated for an additional 26 hr. Water was added to the cooled reaction mixture and the mixture was stirred for 3 hr. The resulting solid was collected and washed with water, then was taken up in ethanol and evaporated to dryness under vacuum. The residual solid was slurried in ether, then collected on a filter and washed with additional ether to provide the title compound (506 mg, 43%) as a yellow solid.

### Compound 1-4 (alternate synthesis): 2-(3,5-Dimethyl-pyrazol-1-yl)-6-(4-methoxy-3,4,5,6-tetrahydro-2H-[1,2′]bipyridinyl-6′-yl)-pyrimidin-4-ylamine

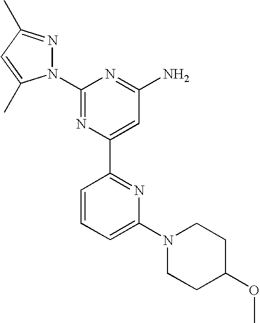

Compound 1-4 was prepared by the methods used for the synthesis of Intermediates 9-12 and for the synthesis of Compound 1-20 (alternate method), starting with Intermediate 16 in place of Intermediate 8.

### Compound 1-7 (alternate synthesis): N-[2-(3,5-Dimethyl-pyrazol-1-yl)-6-(6-morpholin-4-yl-pyridin-2-yl)-pyrimidin-4-yl]-acetamide

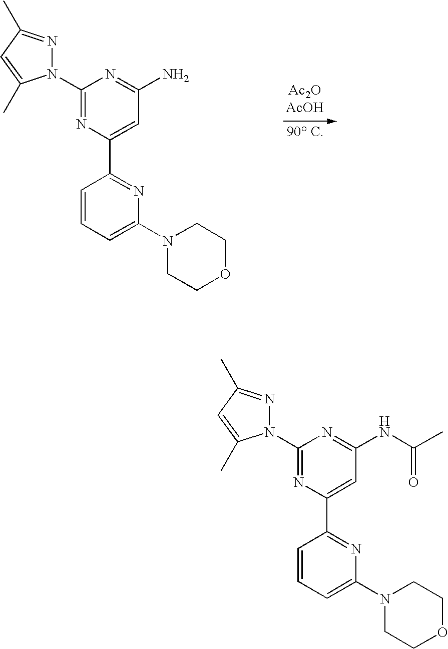

Compound 1-7 was prepared according to the procedures described in the alternative synthesis of Compound 1-3, except that Compound 1-20 was used instead of Compound 1-4.

### Compound 4-1: 2-(Pyridin-2-yl)-6-(6-morpholin-4-yl-pyridin-2-yl)-pyrimidin-4-yl-amine

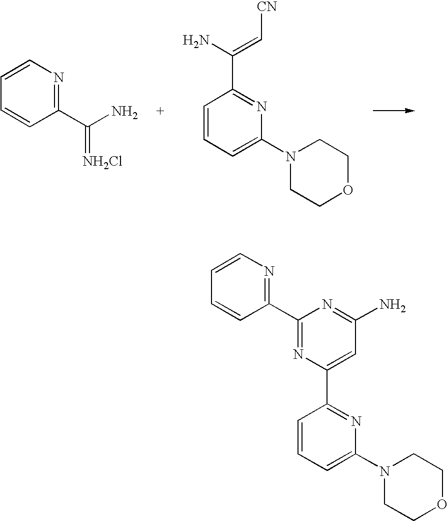

A mixture of Intermediate 9 (230 mg, 1.0 mmol), pyridine-2-carboxamidine hydrochloride (302 mg, 2.0 mmol), sodium ethoxide solution (21 wt %, Aldrich, 0.98 ml, 3.0 mmol), and ethanol (1 ml) was heated with stirring in a microwave reactor at 160° C. for 30 min. Additional sodium ethoxide solution (0.5 ml, 1.5 eq) was added and the mixture was subjected to additional heating in the microwave reactor at 180° C. for 20 min. The solvent was evaporated, then water (2 ml) was added and the mixture was extracted with ethyl acetate. The combined organic extracts were concentrated, then the residue was purified by HPLC to provide the title compound as a TFA salt. LCMS (Method 1) m/z 334.9 [MH+], Tr=4.17 min. TFA salt

### Compound 4-2: 2-(5-methylfuran-2-yl)-6-(6-morpholin-4-yl-pyridin-2-yl)-pyrimidin-4-yl-amine

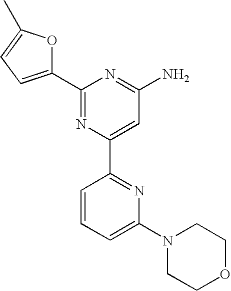

Compound 4-2 was prepared according to the procedures described in Compound 4-1, except that Intermediate 17 was used instead of pyridine-2-carboxamidine hydrochloride. LCMS (Method 1) m/z 337.7 [MH+], Tr=7.87 min.

### Compound 5-1: N-[6-(6-Morpholin-4-yl-pyridin-2-yl)-2-pyridin-2-yl-pyrimidin-4-yl]-acetamide

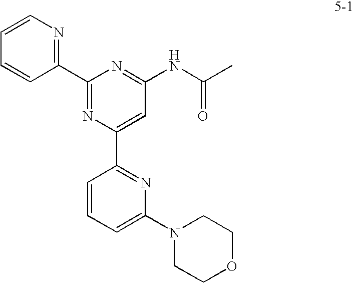

Acetyl chloride (0.027 ml, 0.38 mmol) was added to a solution of Compound 4-1 (100 mg, 0.30 mmol) and pyridine (0.080 ml, 1.0 mmol) in dichloromethane (2 ml) at 0° C. The reaction mixture was stirred and allowed to warm to rt over 16 hr. The mixture was concentrated, and the residue was purified by preparative thin layer chromatography (9:1 dichloromethane/methanol eluant) to provide the title compound (10 mg).

### Compound 5-2: N-[2-(5-Methyl-furan-2-yl)-6-(6-morpholin-4-yl-pyridin-2-yl)-pyrimidin-4-yl]-acetamide

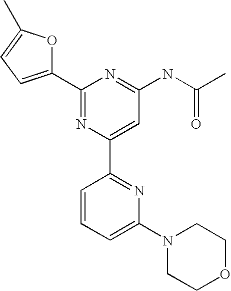

Compound 5-2 was prepared according to the procedures described in Compound 5-1, except that Compound 4-2 was used instead of Compound 4-1.

### Compound 5-3: N-[2-(3,5-Dimethyl-pyrazol-1-yl)-6-(4-morpholin-4-yl-pyridin-2-yl)-pyrimidin-4-yl]-acetamide

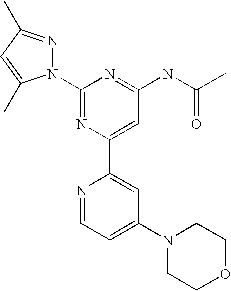

Compound 5-3 was prepared according to the procedures described in Compound 5-1, except that Compound 3-1 was used instead of Compound 4-1.

TABLE 3

| Cmpd | | MS | Reten | HPLC | |
|---|---|---|---|---|---|
| No. | MW | ION | time (min) | Method | Salt |
| 5-1 | 376.4 | 376.9 | 4.21 | 1 | TFA |
| 5-2 | 379.4 | 379.9 | 6.42 | 1 | TFA |
| 5-3 | 393.4 | 393.9 | 3.99 | 1 | Free |
| | | | | | Base |

### Compound 6-1: 2-(3,5-Dimethyl-pyrazol-1-yl)-6-(6-morpholin-4-yl-pyridin-2-yl)-pyrimidin-4-yl]-carbamic acid methyl ester

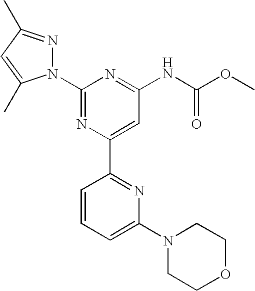

A mixture of Compound 1-20 (200 mg, 0.57 mmol), pyridine (0.27 ml, 3.35 mmol), and dichloromethane (10 ml) was treated with methyl chloroformate (0.114 ml, 1.5 mmol) in portions over 48 hr. Little conversion of starting material was observed (LCMS, TLC). Triphosgene (100 mg, 0.33 mmol) was added and the dark mixture was stirred for 1 hr at rt. Methanol (1 ml) was added. The mixture was stirred for 5 min, then aqueous sodium bicarbonate solution was added and the mixture was extracted with dichloromethane. The combined organic extracts were dried over sodium sulfate, filtered, concentrated, and the residue was purified by silica gel:chromatography (2:1 hexanes/acetone eluant). Trituration (hexanes/ethyl acetate 3:1) provided the title compound (91 mg, 39%) as a yellow solid. LCMS (Method 1) m/z 409.8 [MH+], Tr=6.42.

### Compound 7-1: N-[2-(3,5-Dimethyl-pyrazol-1-yl)-6-(3-morpholin-4-yl-phenyl)-pyrimidin-4-yl]-acetamide

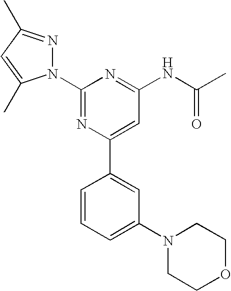

Compound 7-1 was prepared according to the procedure used for Intermediate 3, substituting 3-morpholinophenylboronic acid pinacol ester in place of 6-chloropyridin-2-boronic acid pinacol ester. HPLC purification provided the TFA salt: LCMS (Method 1) m/z 392.9 [MH+], Tr=6.04.

### Compound 8-1: N-[6-(4-Amino-pyridin-2-yl)-2-(3,5-dimethyl-pyrazol-1-yl)-pyrimidin-4-yl]-acetamide

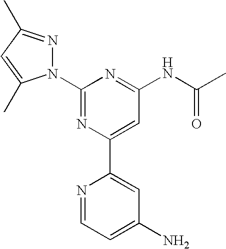

Compound 8-1 was prepared according to the procedure used for Intermediate 3, substituting 4-aminopyridin-2-ylboronic acid pinacol ester in place of 6-chloropyridin-2-boronic acid pinacol ester. HPLC purification provided the TFA salt: LCMS (Method 1) m/z 323.9 [MH+], Tr=3.31.

### Compound 9-1: N-[6-(6-Amino-pyridin-2-yl)-2-(3,5-dimethyl-pyrazol-1-yl)-pyrimidin-4-yl]-acetamide

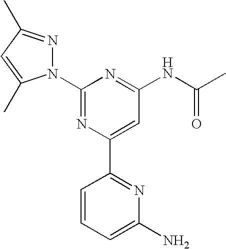

Compound 9-1 was prepared according to the procedure used for Intermediate 3, substituting 6-aminopyridin-2-ylboronic acid pinacol ester in place of 6-chloropyridin-2-boronic acid pinacol ester. HPLC purification provided the TFA salt: LCMS (Method 1) m/z 323.9 [MH+], Tr=3.65.

### Intermediate 18: N-[2-(3,5-Dimethyl-pyrazol-1-yl)-6-(3-fluoro-5-hydroxy-phenyl)-pyrimidin-4-yl]-acetamide

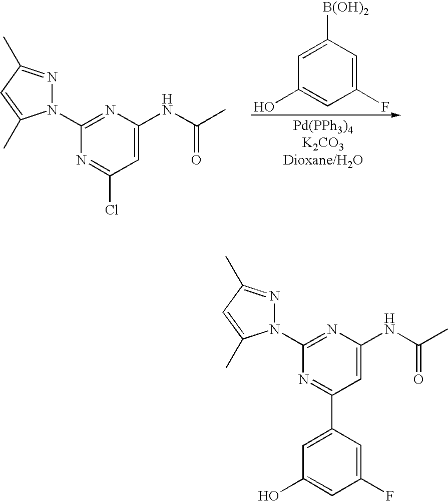

To a solution of Intermediate 2 (5.7 mmol, 1.5 g) in dioxane (30 mL) and water (3 mL) was added (3-fluoro-5-hydroxyphenyl)boronic acid (8.5 mmol, 1.3 g) and potassium carbonate (6.8 mmol, 950 mg). The solution was degassed with nitrogen for 5 minutes. Tetrakis(triphenylphosphine)palladium(0) (0.06 mmol, 60 mg) was added and the mixture was heated at 100° C. for 6 hours. Once cool, the reaction was diluted with water and filtered. The solid filter cake was triturated with ether (100 mL) to yield the title compound (1.7 g, 87%) as a light brown solid. LCMS (Method 3) m/z 341.8 [MH+], Tr=2.57 min.

### Compound 10-1: N-{2-(3,5-Dimethyl-pyrazol-1-yl)-6-[3-fluoro-5-(2-morpholin-4-yl-ethoxy)-phenyl]-pyrimidin-4-yl}-acetamide

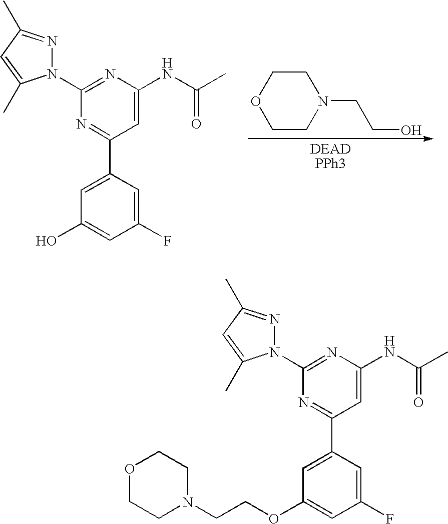

To a solution of Intermediate 18 (1.5 mmol, 0.5 g) in anhydrous tetrahydrofuran (4 mL) was added triphenylphosphine (2.2 mmol, 576 mg) followed by N-(2-hydroxyethyl)morpholine (2.2 mmol, 288 mg) and diethyl azodicarboxylate (2.2 mmol, 382 mg). The mixture was left to stir at room temperature for 16 hr. The completed reaction was neutralized with saturated NaHCO₃ (aq.) and diluted with H₂O. The product was extracted with dichloromethane (3×25 mL). The combined organic layers were dried over magnesium sulfate, filtered and the solvent removed under vacuum. The residue was purified by silica gel chromatography (5% methanol+1% triethylamine in dichloromethane as eluant) to afford the title compound (50%) as a fine white powder. LCMS (Method 3) m/z 454.9 [MH+], Tr=4.61 min.

The compounds of Table 4 were prepared from Intermediate 18 according to the procedure of Compound 10-1, substituting the appropriate alcohol in place of N-(2-hydroxyethyl)morpholine. The products were purified by HPLC/MS using 5-65% acetonitrile in water (0.05% TFA) to give the final compounds as TFA salts.

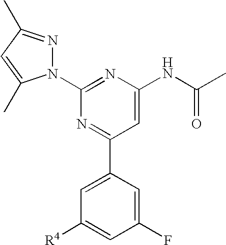

TABLE 4

| Cmpd | | | MS | Reten | HPLC |
|---|---|---|---|---|---|
| No. | R⁴ | MW | ION | time (min) | Method |
| 10-1 | morpholin-4-yl- | 454.5 | 454.9 | 4.61 | 1 |
| | ethoxy | | | | |
| 10-2 | (R)-1-methyl- | 424.5 | 424.8 | 4.54 | 1 |
| | pyrrolidin-3-yloxy | | | | |
| 10-3 | (S)-1-methyl- | 424.5 | 424.8 | 4.56 | 1 |
| | pyrrolidin-3-yloxy | | | | |
| 10-4 | pyrrolidin-1-yl- | 438.5 | 438.9 | 4.57 | 1 |
| | ethoxy | | | | |
| 10-5 | methoxy-ethoxy | 399.4 | 399.9 | 6.88 | 1 |
| 10-6 | dimethylamino- | 412.5 | 412.9 | 4.39 | 1 |
| | ethoxy | | | | |
| 10-7 | 1-methyl-pyrrolidin- | 424.5 | 424.9 | 4.50 | 1 |
| | 3-yloxy | | | | |
| 10-8 | 1-methyl-piperidin- | 438.5 | 438.9 | 4.67 | 1 |
| | 3-yloxy | | | | |
| 10-9 | 1-methyl-piperidin- | 438.5 | 438.9 | 4.79 | 1 |
| | 4-yloxy | | | | |
| 10-10 | 1-isopropyl- | 452.5 | 452.9 | 4.83 | 1 |
| | pyrrolidin- | | | | |
| | 3-yloxy | | | | |
| 10-11 | 1-ethyl-pyrrolidin- | 438.5 | 438.9 | 4.71 | 1 |
| | 3-yloxy | | | | |

### Compound 10-12: (S)-3-{3-[6-Acetylamino-2-(3,5-dimethyl-pyrazol-1-yl)-pyrimidin-4-yl]-5-fluoro-phenoxy}-pyrrolidine-1-carboxylic acid tert-butyl ester

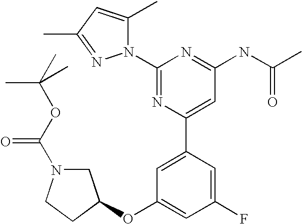

To a solution of Intermediate 18 (8.8 mmol, 3.0 g) in anhydrous tetrahydrofuran (30 mL) was added triphenylphosphine (13.0 mmol, 3.4 g) followed by (3R)-(−)-N-Boc-3-hydroxypyrrolidine (13.0 mmol, 2.5 g) and diethyl azodicarboxylate (13.0 mmol, 2.3 g). The mixture was left to stir at rt for 16 hr. The completed reaction was neutralized with saturated NaHCO₃ (aq.) and diluted with H₂O. The product was extracted with dichloromethane (3×100 mL). The combined organic layers were dried over magnesium sulfate, filtered and the solvent removed under vacuum. The residue was purified by silica gel column (5% methanol+1% triethylamine in dichloromethane as eluant) to afford the title compound (30%) as a yellow oil. Compound 10-12 was used without further purification to make compound 1-13. A sample was further purified by HPLC/MS using 15-75% acetonitrile in water (0.05% TFA). LCMS (Method 1) m/z 511.0 [MH+], Tr=8.43 min.

### Compound 10-13: N-{2-(3,5-Dimethyl-pyrazol-1-yl)-6-[3-fluoro-5-((S)-pyrrolidin-3-yloxy)-phenyl]-pyrimidin-4-yl}-acetamide

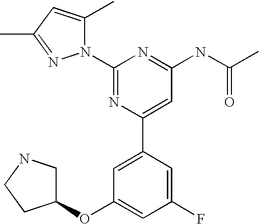

To a solution of Compound 10-12 (1.6 mmol, 810 mg) in ether (10 mL) was added 2N HCl in ether (4.8 mmol, 2.4 mL). The mixture was stirred at room temperature for 1 hr. The resulting precipitate was filtered off, washed with ether and dried under vacuum to yield the title compound (95%) as a fine white solid. A sample was further purified by HPLC/MS using 15-75% acetonitrile in water (0.05% TFA) to yield the final compound as a TFA salt. LCMS (Method 1) m/z 410.9 [MH+], Tr=4.48 min.

### Compound 10-14: N-(2-(3,5-Dimethyl-pyrazol-1-yl)-6-{3-fluoro-5-[(S)-1-(2-methoxy-ethyl)-pyrrolidin-3-yloxy]-phenyl}-pyrimidin-4-yl)-acetamide

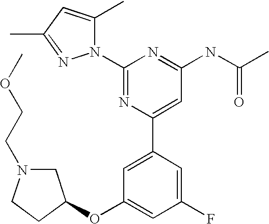

To a solution of compound 10-13 (0.24 mmol, 100 mg) in anhydrous DMF (1.5 mL) was added potassium carbonate (0.56 mmol, 77 mg) and 2-bromo-1-methoxyethane (0.31 mmol, 43 mg). The reaction mixture was stirred at 50° C. for 12 hours. Once cool, the reaction was diluted with methanol, filtered and purified by HPLC/MS using 15-75% acetonitrile in water (0.05% TFA) to yield the title compound as a TFA salt. LCMS (Method 1) m/z 469.0 [MH+], Tr=4.75 min.

### Intermediate 19: N-[2-(3,5-Dimethyl-pyrazol-1-yl)-6-(3-hydroxy-phenyl)-pyrimidin-4-yl]-acetamide

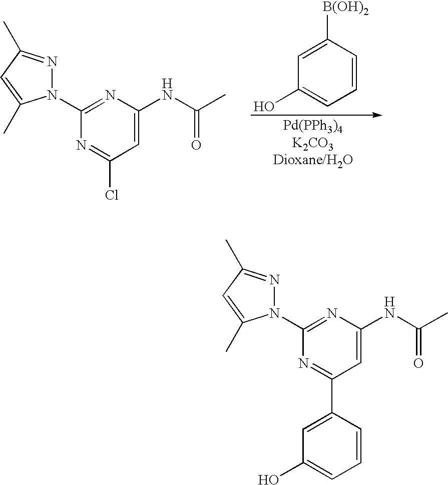

Intermediate 19 was prepared according to the procedure of Intermediate 18 using 3-hydroxyphenylboronic acid in place of (3-fluoro-5-hydroxyphenyl)boronic acid. LCMS (Method 3) m/z 323.8 [MH+], Tr=2.44 min.

The compounds of Table 5 were prepared according to the procedure of Compound 10-1, substituting Intermediate 19 in place of Intermediate 18, and using the appropriate alcohol in place of N-(2-hydroxyethyl)morpholine. Purification by HPLC/MS using 5-65% acetonitrile in water (0.05% TFA) gave the final compounds as TFA salts.

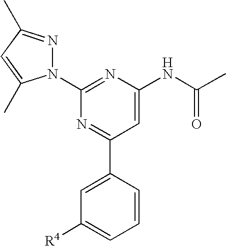

TABLE 5

| Cmpd | | | MS | Reten | HPLC |
|---|---|---|---|---|---|
| No. | R⁴ | MW | ION | time (min) | Method |
| 11-1 | 1-methyl-pyrrolidin- | 406.5 | 406.9 | 7.79 | 2 |
| | 3-yloxy | | | | |
| 11-2 | (R)-1-methyl- | 406.5 | 407.0 | 4.37 | 1 |
| | pyrrolidin-3-yloxy | | | | |
| 11-3 | (S)-1-methyl- | 406.5 | 407.3 | 4.20 | 1 |
| | pyrrolidin-3-yloxy | | | | |
| 11-4 | 2-pyrrolidin-1-yl- | 420.5 | 420.9 | 4.26 | 1 |
| | ethoxy | | | | |
| 11-5 | 2-dimethylamino- | 394.5 | 394.9 | 4.45 | 1 |
| | ethoxy | | | | |
| 11-6 | 2-morpholin-4-yl- | 436.5 | 436.9 | 4.48 | 1 |
| | ethoxy | | | | |
| 11-7 | 1-methyl-piperidin-3- | 420.5 | 420.9 | 4.55 | 1 |
| | yloxy | | | | |
| 11-8 | 1-methyl-piperidin-4- | 420.5 | 420.9 | 4.42 | 1 |
| | yloxy | | | | |
| 11-9 | 1-isopropyl- | 434.5 | 434.9 | 7.79 | 2 |
| | pyrrolidin-3-yloxy | | | | |
| 11-10 | 1-ethyl-pyrrolidin-3- | 420.5 | 420.9 | 4.37 | 1 |
| | yloxy | | | | |

### Intermediate 20: N-[2-(3,5-Dimethyl-pyrazol-1-yl)-6-(2-hydroxy-phenyl)-pyrimidin-4-yl]-acetamide

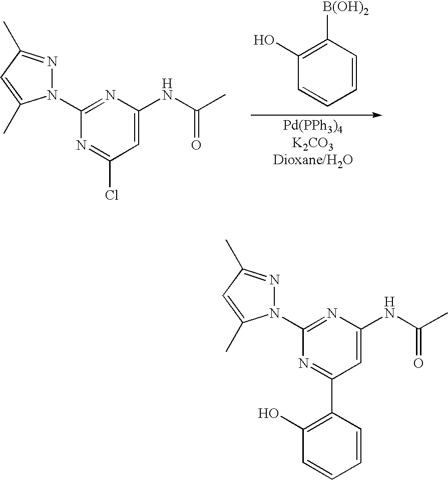

Intermediate 20 was prepared according to the procedure of Intermediate 18, substituting 2-hydroxyphenylboronic acid in place of (3-fluoro-5-hydroxyphenyl)boronic acid. LCMS (Method 3) m/z 323.8 [MH+], Tr=2.85 min.

The compounds of Table 6 were prepared according to the procedure of Compound 10-1, substituting Intermediate 20 in place of Intermediate 18, and using the appropriate alcohol in place of N-(2-hydroxyethyl)morpholine. Purification by HPLC/MS using 5-65% acetonitrile in water (0.05% TFA) gave the final compounds as TFA salts.

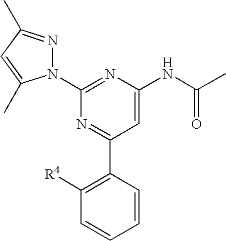

TABLE 6

| Cmpd | | | MS | Reten | HPLC |
|---|---|---|---|---|---|
| No. | R⁴ | MW | ION | time (min) | Method |
| 12-1 | 2-methoxy-ethoxy | 381.4 | 381.9 | 6.44 | Method 1 |
| 12-2 | 2-pyrrolidin-1-yl- | 420.5 | 421.4 | 4.64 | Method 1 |
| | ethoxy | | | | |
| 12-3 | 2-dimethylamino- | 394.5 | 395.3 | 4.56 | Method 1 |
| | ethoxy | | | | |

### Intermediate 21: N-[2-(3,5-Dimethyl-pyrazol-1-yl)-6-(3-hydroxymethyl-phenyl)-pyrimidin-4-yl]-acetamide

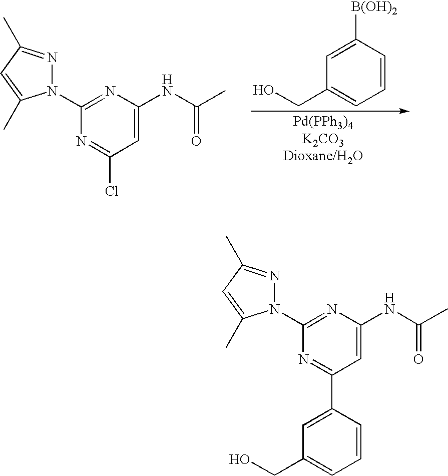

Intermediate 21 was prepared according to the procedure of Intermediate 18, substituting 3-hydroxymethylphenylboronic acid in place of (3-fluoro-5-hydroxyphenyl)boronic acid, yielding the title compound (60%) as a as a fine white solid. LCMS (Method 3) m/z 337.9 [MH+], Tr=2.39 min.

### Intermediate 22: Methanesulfonic acid 3-[6-acetylamino-2-(3,5-dimethyl-pyrazol-1-yl)-pyrimidin-4-yl]-benzyl ester

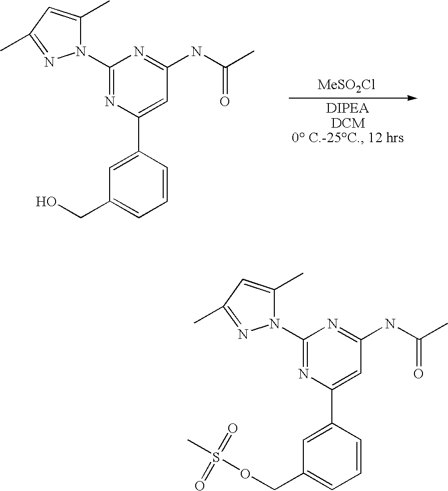

To a solution of Intermediate 21 (3.0 mmol, 1.0 g) in anhydrous dioxane (15 mL) was added diisopropylethylamine (4.5 mmol, 580 mg). The reaction mixture was stirred under nitrogen in a 0° C. ice bath and methanesulfonyl chloride (4.5 mmol, 570 mg) was added. The reaction mixture was stirred at room temperature for 12 hours. The completed reaction was neutralized with saturated NaHCO₃ (aq.) and diluted with H₂O. The product was extracted with dichloromethane (3×100 mL). The combined organic layers were dried over magnesium sulfate, filtered and the solvent removed under vacuum to yield the title compound (60%) as a pale yellow foam. LCMS (Method 3) m/z 415.9 [MH+], Tr=2.217 min.

### Compound 13-1: N-{2-(3,5-Dimethyl-pyrazol-1-yl)-6-[3-((R)-1-methyl-pyrrolidin-3-yloxymethyl)-phenyl]-pyrimidin-4-yl}-acetamide

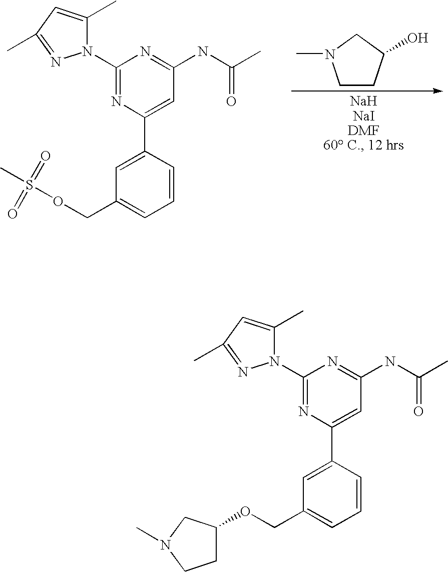

To a solution of Intermediate 22 (0.72 mmol, 300 mg) in anhydrous DMF (2 mL) was added sodium iodide (0.72 mmol, 108 mg). The reaction mixture was stirred at room temperature for ten minutes. A solution of (R)-(+1-methyl-3-hydroxypyrrolidine (1.1 mmol, 110 mg) in anhydrous DMF (1.0 mL) and sodium hydride (0.72 mmol, 30 mg) was then added. The reaction mixture was stirred at 60° C. for 12 hr. Once cool, the reaction was diluted with methanol, filtered and purified by HPLC/MS using 15-75% acetonitrile in water (0.05% TFA) to yield the title compound (20%). LCMS (Method 1) m/z 421.2 [MH+], Tr=5.57 min.

### Compound 13-2: N-{2-(3,5-Dimethyl-pyrazol-1-yl)-6-[3-((S)-1-methyl-pyrrolidin-3-yloxymethyl)-phenyl]-pyrimidin-4-yl}-acetamide

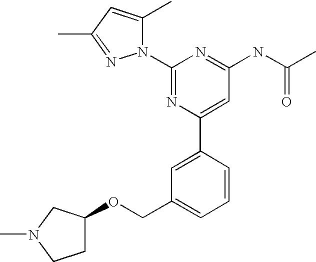

Compound 13-2 was prepared according to the procedure of Compound 13-1, substituting (S)-(+)-1-methyl-3-hydroxypyrrolidine for (R)-(−)-1-methyl-3-hydroxypyrrolidine in place of (R)-(−)-1-methyl-3-hydroxypyrrolidine, to yield the title compound (20%). LCMS (Method 1) m/z 421.2 [MH+], Tr=5.55 min.

### Intermediate 23: N-[2-(3,5-Dimethyl-pyrazol-1-yl)-6-(3-formyl-phenyl)-pyrimidin-4-yl]-acetamide

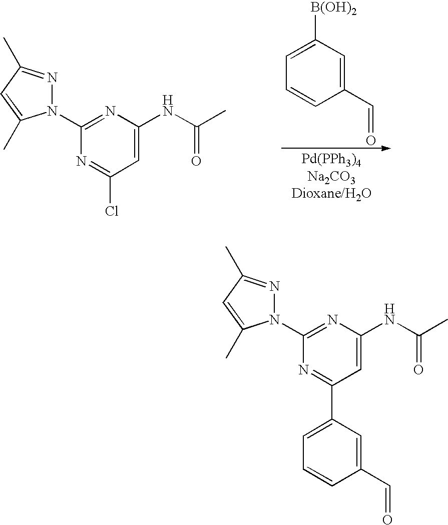

A mixture of Intermediate 2 (1.3 g, 4.0 mmol), 3-formylphenylboronic acid (0.9 g, 6.0 mmol) and sodium carbonate (1.7 g, 16.0 mmol) in dioxane/water (9/1, 30 mL) was degassed with bubbling N₂ for 15 min.

Tetrakis(triphenylphosphine)palladium(0) (0.46 g, 0.4 mmol) was added and the mixture was heated at 90° C. for 16 hr. The solution was poured into water (30 mL) and extracted with ethyl acetate (2×30 mL). The organic layer was washed with water (2×15 mL) and brine (15 mL), dried (Na₂SO₄), and the solvent removed under reduced pressure. The residue was purified by chromatography on silica gel (1:2 acetone/DCM as eluant) to provide the title compound. LCMS (Method 3) m/z 335.8 [MH+], Tr=2.53 min.

The compounds of Table 7 were prepared according to the following procedure: A solution of Intermediate 23 (50 mg, 0.15 mmol) and the appropriate amine (0.30 mmol, 2 eq) in methanol (1 ml) was stirred for 5 min, then pyridine-borane complex (0.3 mmol, 2 eq) was added. The mixture was stirred at rt for 16 hr Purification by HPLC/MS using 15-75% acetonitrile in water (0.05% TFA) yielded the final product as TFA salt.

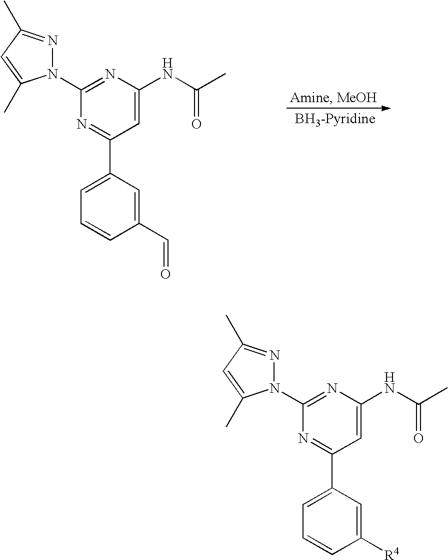

TABLE 7

| Cmpd. | | | MS | Retention | HPLC |
|---|---|---|---|---|---|
| No. | R⁴ | MW | ION | time (min) | Method |
| 14-1 | Pyrrolidin-1-yl-methyl | 390.5 | 390.9 | 4.05 | 2 |
| 14-2 | N,N-dimethylamino- | 364.4 | 364.9 | 3.82 | 2 |
| | methyl | | | | |
| 14-3 | N-(2- | 394.5 | 394.9 | 4.02 | 2 |
| | methoxyethyl)amino- | | | | |
| | methyl | | | | |
| 14-4 | Morpholin-4-yl-methyl | 406.5 | 406.9 | 3.86 | 2 |
| 14-5 | 4-methylpiperazin-1-yl- | 419.5 | 419.9 | 3.62 | 2 |
| | methyl | | | | |
| 14-6 | N-methyl-N-(2- | 408.5 | 408.9 | 4.39 | 2 |
| | methoxyethyl)amino- | | | | |
| | methyl | | | | |
| 14-7 | (R)-2-methoxymethyl- | 434.5 | 434.9 | 4.44 | 2 |
| | pyrrolidin-1-yl-methyl | | | | |
| 14-8 | (S)-2-methoxymethyl- | 434.5 | 434.9 | 4.45 | 2 |
| | pyrrolidin-1-yl-methyl | | | | |

### Intermediate 24: N-[2-(3,5-Dimethyl-pyrazol-1-yl)-6-(2-fluoro-5-formyl-phenyl)-pyrimidin-4-yl]-acetamide

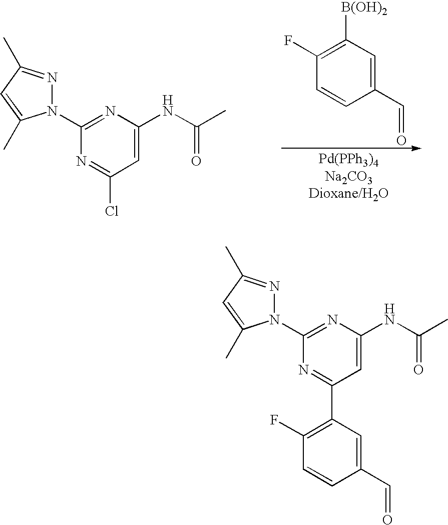

Intermediate 24 was prepared according to the procedure of Intermediate 23, substituting 2-fluoro-5-formylphenylboronic acid in place of 3-formylphenylboronic acid. LCMS (Method 3) m/z 353.8 [MH+], Tr=2.557 min.

### Compound 15-1: N-[2-(3,5-Dimethyl-pyrazol-1-yl)-6-(2-fluoro-5-pyrrolidin-1-ylmethyl-phenyl)-pyrimidin-4-yl]-acetamide

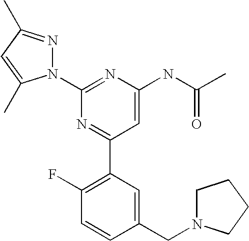

Compound 15-1 was prepared according to the procedure of Compounds 14-x (Table 7) LCMS (Method 2) m/z 408.9 [MH+], Tr=4.39 min. LCMS (Method 2) m/z 408.9 [MH+], Tr=4.39 min.

### Intermediate 25: N-[2-(3,5-Dimethyl-pyrazol-1-yl)-6-(2-methoxy-5-formyl-phenyl)-pyrimidin-4-yl]-acetamide

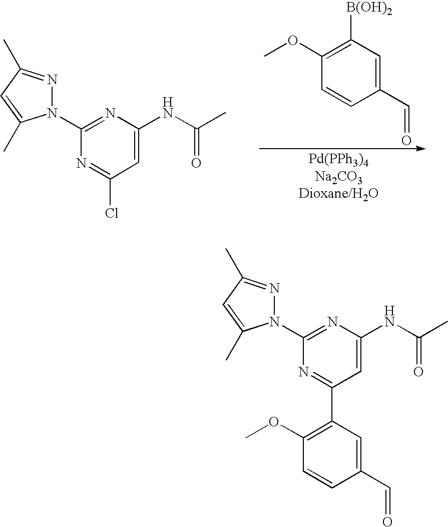

Intermediate 25 was prepared according to the procedure of Intermediate 23, substituting 2-methoxy-5-formylphenylboronic acid in place of 3-formylphenylboronic acid. LCMS (Method 3) m/z 365.8 [MH+], Tr=2.491 min.

### Compound 16-1: N-[2-(3,5-Dimethyl-pyrazol-1-yl)-6-(2-methoxy-5-pyrrolidin-1-ylmethyl-phenyl)-pyrimidin-4-yl]-acetamide

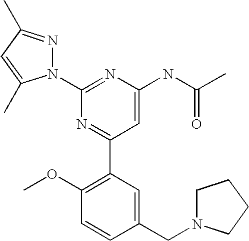

Compound 16-1 was prepared according to the procedure of Compounds 14-x (Table 7). Purification by HPLC/MS using 15-75% acetonitrile in water (0.05% TFA) yielded the title compound as a TFA salt. LCMS (Method 2) m/z 420.9 [MH+], Tr=4.09 min.

### Compound 17-1: N-[2-(3,5-Dimethyl-pyrazol-1-yl)-6-isoquinolin-5-yl-pyrimidin-4-yl]-acetamide

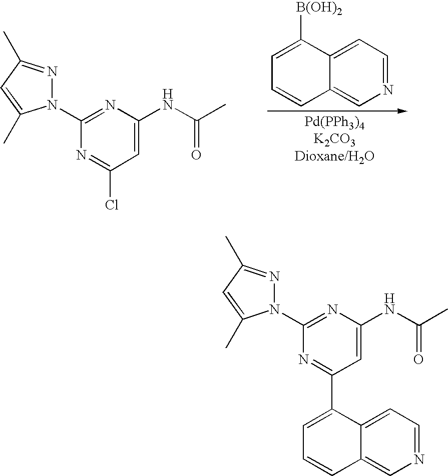

Compound 17-1 was prepared according to the procedure of Intermediate 23, substituting isoquinoline-4-boronic acid in place of 3-formylphenylboronic acid. Purification by HPLC/MS yielded the title compound (240 mg, 59%) as an off white solid. LCMS (Method 1) m/z 358.8 [MH+], Tr=3.80 min.

### Compounds 18-1 and 19-1: N-[2-(3,5-Dimethyl-pyrazol-1-yl)-6-(1,2,3,4,4a,8a-hexahydro-isoquinolin-5-yl)-pyrimidin-4-yl]-acetamide and N-[2-(3,5-Dimethyl-pyrazol-1-yl)-6-(2-ethyl-1,2,3,4,4a,8a-hexahydro-isoquinolin-5-yl)-pyrimidin-4-yl]-acetamide

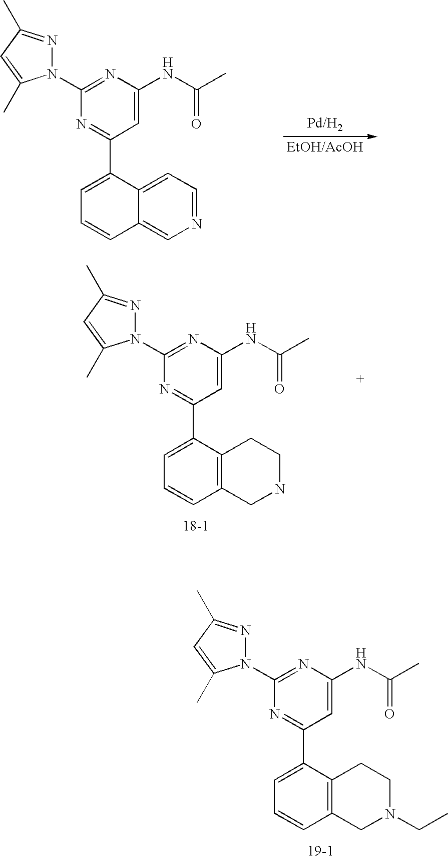

A solution of Compound 17-1 (200 mg) in acetic acid (10 mL) and EtOH (5 mL) was hydrogenated at 1 atmosphere pressure over a 10% Pd/C catalyst for 48 hr. The filtered reaction mixture was concentrated and purified by preparative silica gel TLC (90:9:1 chloroform:MeOH: aqueous ammonia as eluant) to give Compounds 18-1 and 19-1. Compound 18-1: white solid; LCMS (Method 1) m/z 363.0 [MH+], Tr=3.73 min. Compound 19-1: white solid; LCMS (Method 2) m/z 390.9 [MH+], Tr=3.83 min.

### Compound 20-1: N-[2-(3,5-Dimethyl-pyrazol-1-yl)-6-(2-methyl-1,2,3,4,4a,8a-hexahydro-isoquinolin-5-yl)-pyrimidin-4-yl]-acetamide

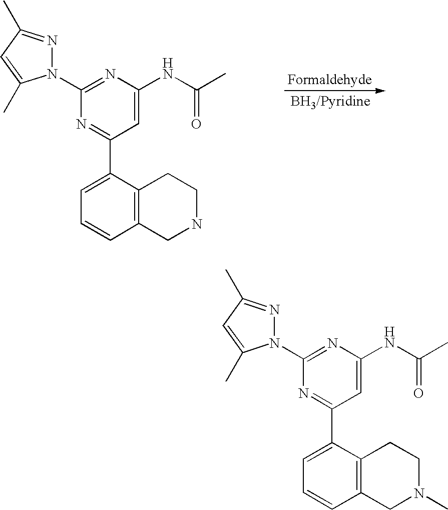

A solution of Compound 18-1 (100 mg, 0.28 mmol) in EtOH (1 mL) and acetic acid (0.05 ml) was treated with 37% aqueous formaldehyde solution (0.4 ml), followed by pyridine-borane complex (0.2 ml, 0.2 mmol). The reaction mixture was stirred at room temperature for 12 hr. The reaction mixture was purified by HPLC/MS using 5-65% acetonitrile in water (0.05% TFA) to give the title compound as a TFA salt. LCMS (Method 1) m/z 376.9 [MH+], Tr=3.71 min.

### Intermediate 26: 7-Bromo-3,4-dihydro-1H-isoquinoline-2-carboxylic acid tert-butyl ester

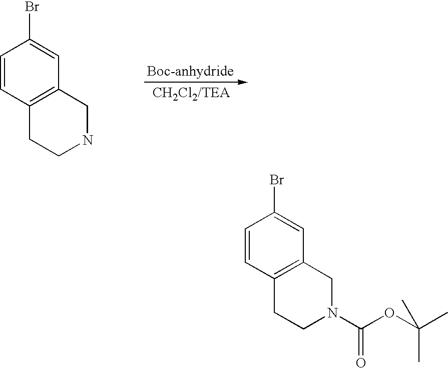

To a solution of 7-Bromo-1,2,3,4-Tetrohedro-isoquinoline (4.0 g 18.9 mmol, 1 eq) in dichloromethane (20 ml) were added di-t-butyl dicarbonate (4.9 g, 22.5 mmol) and triethylamine (4 ml, 36.4 mmol). The reaction mixture was stirred at rt for 16 hr. Aqueous sodium bicarbonate solution (150 ml) was added and the mixture was extracted with dichloromethane. The organic layer was dried over sodium sulfate, filtered, and concentrated, then the residue was purified by silica gel chromatography (0-20% ethyl acetate in hexanes as eluant). Intermediate 26 was obtained as a clear oil (3.8 g, 64.6%). LCMS (Method 2) m/z 211.7 [MH+], Tr=3.18 min.

### Intermediate 27: 7-(4,4,5,5-Tetramethyl-[1,3,2]dioxaborolan-2-yl)-3,4-dihydro-1H-isoquinoline-2-carboxylic acid tert-butyl ester

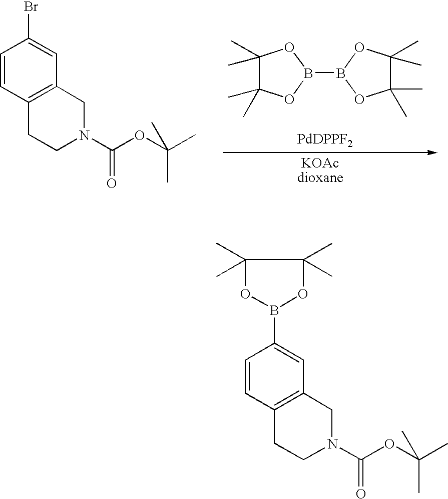

A mixture of Intermediate 26 (2.6 g, 8.3 mmol), bis-pinacolatodiboron (2.54 g, 10.0 mmol), potassium acetate (1.63 g, 16.6 mmol) and Pd(dpff)₂ (0.68 g, 0.83 mmol) in dioxane (30 ml) was stirred in a sealed tube at 100° C. for 16 hr. Aqueous sodium bicarbonate solution (100 ml) was added to the cooled reaction mixture and the mixture was extracted with dichloromethane. The organic layer was dried over sodium sulfate, filtered, and concentrated, then the residue was purified by silica gel chromatography (10% ethyl acetate in hexanes as eluant). Intermediate 27 was obtained as a slightly yellow solid (2.2 g, 74%). LCMS (Method 2) m/z 259.9 [MH+], Tr=3.35 min.

### Intermediate 28: 7-[6-Acetylamine-2-(3,5-dimethyl-pyrazol-1-yl)-pyrimidin-4-yl]-3,4-dihydro-1H-isoquinoline-2-carboxylic acid tert-butyl ester

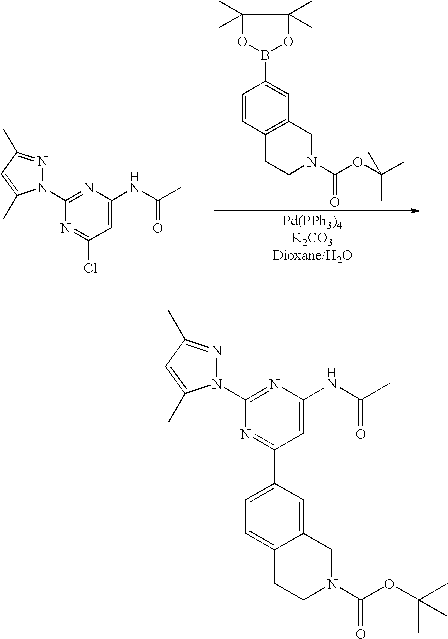

Intermediate 28 was prepared according to the procedure of Intermediate 23, substituting Intermediate 27 in place of 3-formylphenylboronic acid. Purification by silica gel chromatography (1:1 hexanes/ethyl acetate as eluant) yielded the title compound as a white foam (2.2 g, 73%). LCMS (Method 2) m/z 462.9 [MH+], Tr=2.95 min.

### Compound 21-1: N-[2-(3,5-Dimethyl-pyrazol-1-yl)-6-(1,2,3,4-tetrahydro-isoquinolin-7-yl)-pyrimidin-4-yl]-acetamide

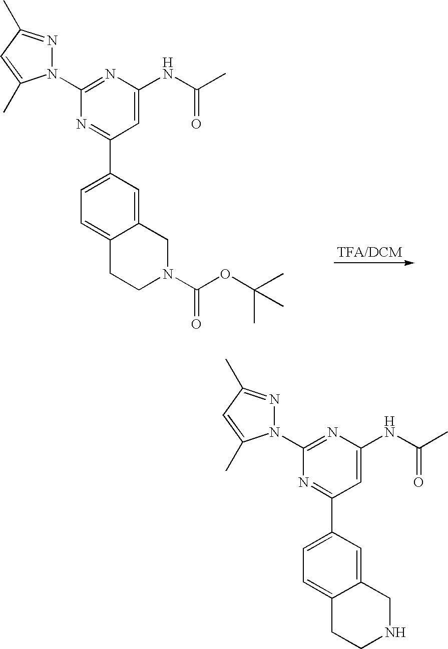

Intermediate 28 (100 mg) was treated with the mixture of 1 ml dichloromethane and 1 ml trifluoroacetic acid for 30 min. The solvents were removed and a sample was purified by prep HPLC/MS to obtain the title compound as a TFA salt. LCMS (Method 1) m/z 362.9 [MH+], Tr=3.80 min.

### Compound 22-1: N-[2-(3,5-Dimethyl-pyrazol-1-yl)-6-(2-methyl-1,2,3,4-tetrahydro-isoquinolin-7-yl)-pyrimidin-4-yl]-acetamide

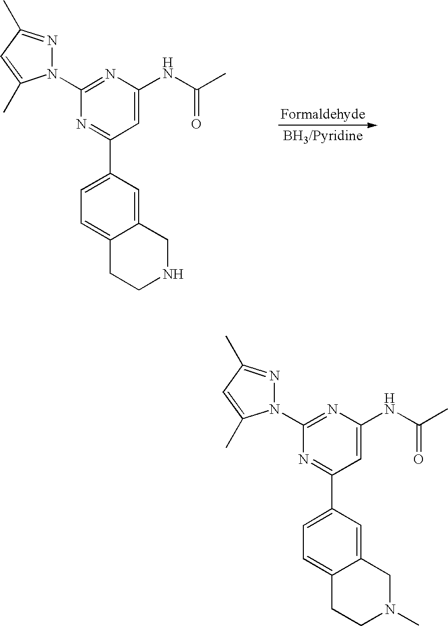

Compound 22-1 was prepared according to the procedure of Compound 20-1, substituting Compound 21-1 (20 mg) in place of Compound 18-1. Purification by preparative HPLC/MS using 5-65% acetonitrile in water (0.05% TFA) gave the title compound as a TFA salt LCMS (Method 1) m/z 376.9 [MH+], Tr=3.76 min.

### Compound 23-1: N-[2-(3,5-Dimethyl-pyrazol-1-yl)-6-(2-ethyl-1,2,3,4-tetrahydro-isoquinolin-7-yl)-pyrimidin-4-yl]-acetamide

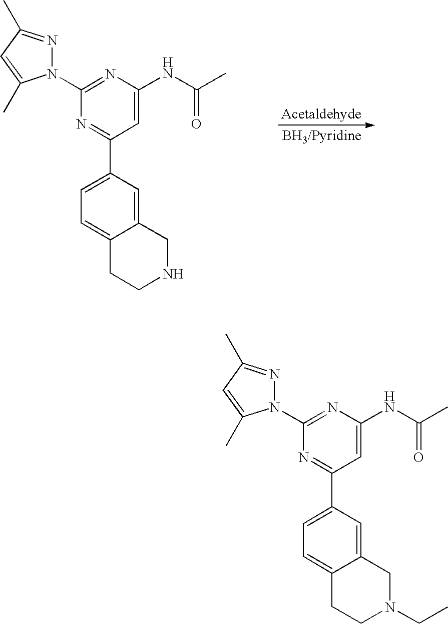

Compound 23-1 was prepared according to the method of Compound 22-1, substituting acetaldehyde in place of formaldehyde solution. LCMS (Method 1) m/z 390.9 [MH+], Tr=3.85 min.

It will be appreciated that, although specific embodiments of the invention have been described herein for purposes of illustration, various modifications may be made without departing from the spirit and scope of the invention. Accordingly, the invention is not limited except as by the appended claims.

What is claimed is:

1. A compound of formula (I) 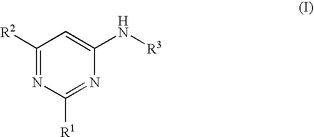
or a pharmaceutically acceptable salt, ester, solvate or stereoisomer thereof, wherein:
R¹ is a heterocycle optionally substituted by one or more members selected from the group of lower alkyl, lower alkoxy, halogen and cyano;
R² is phenyl or pyridyl, wherein the phenyl or pyridyl ring is substituted by 1 to 4 R⁴ groups; or
R² is isoquinoline, dihydroisoquinoline or tetrahydroisoquinoline ring, wherein the isoquinoline, dihydroisoquinoline or tetrahydroisoquinoline ring is optionally substituted by R⁵;
R³ is H, R⁷, COR⁷, CONR⁷R⁸, or COOR⁷;
R⁴ is at each occurrence selected from the group of halogen, —(X)ₘ—(O), —(Y)ₚ—R⁵, or —(X)ₘ—(O)ₙ—(Y)ₚ—NR⁵R⁶;
each of R⁵ and R⁶ is independently hydrogen, lower alkyl, lower C₂₋₆ alkoxy, lower C₂₋₆ alkoxyalkyl, lower C₂₋₆ hydroxyalkyl, cyano, C(O)—C₁-C₆alkyl or C(O)O—C₁-C₆alkyl; or
R⁵ and R⁶ together with the nitrogen to which they are attached form a heterocyclic ring optionally substituted by one or more members selected from the group of halogen, hydroxyl, lower alkyl, lower alkoxy, lower alkoxyalkyl, lower hydroxyalkyl, cyano, and —C(O)—C₁-C₆alkyl;
R⁷ is lower alkyl optionally substituted by one or more members selected from the group of lower alkyl, lower alkoxy, hydroxyl, halogen, amino, alkylamino and dialkylamino;
R⁸ is selected from the group of lower alkyl, lower alkoxy, alkoxyalkyl, —C(O)—C₁-C₆alkyl or lower alkenyl, wherein the lower alkyl, lower alkoxy, alkoxyalkyl, —C(O)—C₁-C₆alkyl, and lower alkenyl groups are optionally substituted by one or more lower alkyl, halogen, lower alkoxy, hydroxyl, or cyano; or
R⁷ and R⁸ together with the nitrogen to which they are attached form a heterocyclic ring optionally substituted by one or more members selected from the group of halogen, hydroxyl, lower alkyl, lower alkoxy, lower alkoxyalkyl, lower hydroxyalkyl and cyano;
each of X and Y is independently lower alkyl, cycloalkyl or saturated heterocyclyl;
m is at each occurrence 0 or 1;
n is at each occurrence 0 or 1; and
p is at each occurrence 0 or 1.

2. A compound according to claim 1, wherein R¹ is selected from the group of pyrazolyl, triazolyl, furanyl, thiazolyl and pyridinyl, wherein the pyrazolyl, triazolyl, furanyl, thiazolyl and pyridinyl groups are optionally substituted by one or more member selected from the group of lower alkyl and halogen.

3. A compound according to claim 2, wherein R¹ is pyrazolyl optionally substituted by two lower alkyl groups or furanyl optionally substituted by one lower alkyl group.

4. A compound according to claim 3, wherein R¹ is selected from the group of pyrazol-1-yl, 3,5-dimethyl-pyrazol-1-yl, furan-2-yl and 5-methyl-furan-2-yl.

5. A compound according to claim 4, wherein R¹ is 3,5-dimethyl-pyrazol-1-yl or 5-methyl-furan-2-yl.

6. A compound according to claim 1, wherein R³ is COR⁷.

7. A compound according to claim 6, wherein R⁷ is lower alkyl.

8. A compound according to claim 7, wherein R⁷ is methyl, ethyl or isopropyl.

9. A compound according to claim 8, wherein R⁷ is methyl.

10. A compound according to claim 1, wherein R² is pyridyl substituted by 1 to 4 R⁴ groups.

11. A compound according to claim 10, wherein R² is pyridyl substituted by 1 R⁴ group.

12. A compound according to claim 11, wherein:
R⁴ is —(X)ₘ—(O)ₙ—(Y)ₚ—R⁵;
m is 0;
n is 0;
p is 1; and
Y is a saturated heterocycle.

13. A compound according to claim 1, wherein R² is phenyl substituted by 1 to 4 R⁴ groups.

14. A compound according to claim 1, wherein R² is phenyl substituted by 2 R⁴ groups.

15. A compound according to claim 14, wherein:
one R⁴ is —(X)ₘ—(O)ₙ—(Y)ₚ—R⁵ wherein
m is 0;
n is 0;
p is 1; and
Y is a saturated heterocycle;
and wherein the other R⁴ is halogen, methyl or methoxy.

16. A pharmaceutical composition comprising a compound according to claim 1 and a pharmaceutically acceptable carrier or diluent.

17. A pharmaceutical composition comprising a compound according to claim 3 and a pharmaceutically acceptable carrier or diluent.

18. A pharmaceutical composition comprising a compound according to claim 7 and a pharmaceutically acceptable carrier or diluent.

19. A method for treating a subject having a condition selected from the group of ischemia, supraventricular arrhythmias, acute renal failure, myocardial reperfusion injury, autoimmune disease, addiction, substance abuse, excessive daytime sleepiness, inflammatory bowel diseases, asthma, diabetes mellitus, obesity, Parkinson disease, Huntington's disease, Alzheimer's disease, dystonia and dyskinesia, comprising to a subject in need thereof a pharmaceutical composition according to claim 16.

20. A method according to claim 19, wherein the condition is Parkinson disease, Huntington's disease, Alzheimer's disease, dystonia or dyskinesia.
